(19) United States
(12) Patent Application Publication
(10) Pub. No.: US 2008/0105961 A1
(43) Pub. Date: May 8, 2008

(54) Ligands of the Molecule Fit (Agt-121) and their Pharmaceutical Use

(76) Inventors: Gregory Royce Collier, Victoria (AU); Kenneth Russell Walder, Victoria (AU); James Leonard Trevaskis, Baton Rouge, LA (US); Janine Susan McMillan, Victoria (AU); Lyndal Jane Bayles, Victoria (AU)

Correspondence Address: KNOBBE MARTENS OLSON & BEAR LLP, 2040 MAIN STREET, FOURTEENTH FLOOR, IRVINE, CA, 92614, US

(21) Appl. No.: 10/592,922

(22) PCT Filed: Mar. 16, 2005
(86) PCT No.: PCT/AU05/00372

§ 371 Date: Dec. 11, 2006

Related U.S. Application Data
(60) Provisional application No. 60/553,823, filed on Mar. 16, 2004.

(51) Int. Cl. H01L 23/02

(52) U.S. Cl. 257/686

(57) ABSTRACT

Identification of molecules which modulate inter alia obesity, anorexia, weight maintenance, inflammation and/or metabolic energy levels in a subject are described which particularly relate to a protein molecule called “FIT”, previously known as “AGT-121”. Ligands of “FIT” as well as antagonists of “FIT”-ligand interaction are proposed to modulate inter alia obesity, anorexia, weight maintenance, inflammation and/or metabolic energy levels in a subject. Methods of treatment and prophylaxis and pharmaceutical compositions useful in modulating inter alia obesity, anorexia, weight maintenance, inflammation and/or metabolic energy levels are also provided.

### BACKGROUND OF THE INVENTION

1. Field of the Invention

The present invention relates generally to the identification of molecules which modulate inter alia obesity, anorexia, weight maintenance, inflammation and/or metabolic energy levels in a subject. More particularly, the present invention provides a molecule referred to herein as “FIT” and ligands thereof and antagonists and agonists of FIT-ligand interaction are proposed to modulate inter alia obesity, anorexia, weight maintenance, inflammation and/or metabolic energy levels in a subject. The present invention further provides methods of treatment and prophylaxis and pharmaceutical compositions useful in modulating inter alia obesity, anorexia, weight maintenance, inflammation and/or metabolic energy levels.

2. Description of the Prior Art

Bibliographic details of references provided in the subject specification are listed at the end of the specification.

Reference to any prior art in this specification is not, and should not be taken as, an acknowledgment or any form of suggestion that this prior art forms part of the common general knowledge in any country.

The increasing sophistication of recombinant DNA technology is greatly facilitating research and development in the medical, veterinary and allied human and animal health fields. This is particularly the case in the investigation of the genetic bases involved in the etiology of certain disease conditions. One particularly significant condition from the stand point of morbidity and mortality is obesity.

Obesity is defined as a pathological excess of body fat and is the result of an imbalance between energy intake and energy expenditure for a sustained period of time. Obesity is the most common metabolic disease found in affluent societies. The prevalence of obesity in these affluent societies is alarmingly high, ranging from 10% to upwards of 50% in some sub-populations (Bouchard, The genetics of Obesity, Boca Raton: CRC Press, 1994). Of particular concern is the fact that the prevalence of obesity appears to be rising consistently in affluent societies and is now increasing rapidly in less prosperous nations as they become more affluent and/or adopt cultural practices similar to those in more affluent countries (Zimmet, Diabetes Care 15: 232-252, 1992). The escalating rates of obesity globally have resulted in the World Health Organisation declaring an obesity epidemic worldwide (World Trade Organisation. Obesity. Preventing and managing the global epidemic. Report of a WHO Consultation on Obesity. Geneva: World Health Organisation, 1998).

In Australia, an AusDiab study estimated that 7.5 million Australians (60%) aged 25 years and over were overweight or obese. Of these, 2.6 million (21%) were obese (BMI>30) (Dunstan et al., Diabetes Res. Clin. Pract. 57: 119-129, 2002). Similarly, the prevalence of obesity in the U.S. increased substantially between 1991 and 1998, increasing from 12% to 18% in Americans during this period (Mokdad et al., JAMA 282(16): 1519-1522, 1999).

The high and increasing prevalence of obesity has serious health implications for both individuals and society as a whole. Obesity is a complex and heterogeneous disorder and has been identified as a key risk indicator of preventable morbidity and mortality. Obesity, for example, increases the risk of a number of other metabolic conditions including Type 2 diabetes mellitus and cardiovascular disease (Must et al., JAMA 282(16): 1523-1529, 1999, Kopelman, Nature 404: 635-643, 2000). Alongside obesity the prevalence of diabetes continues to increase rapidly. The AusDiab survey referred to above estimated that close to 1 million Australians aged 25 years and over have Type 2 diabetes (Dunstan et al., 2002 supra). This represents approximately 7.5% of the population. In the U.S., the number of adults with diabetes increased by 49% between 1991 and 2000 (Marx, Science 686-689, 2002). It has been estimated that about 17 million people in the U.S. have Type 2 diabetes and an equal number are thought to be pre-diabetic (Marx, 2002, supra). In Australia, the annual costs of obesity associated with diabetes and other disease conditions has been conservatively estimated to be AUS$810 million for 1992-93 (National Health and Medical Research Council, Acting on Australia's weight: A strategy for the prevention of overweight and obesity. Canberra: National Health and Medical Research Council, 1996). The direct costs of diabetes and its complications in Australia in 1993-94 were estimated at $681 million, or 2.2% of total health system costs in that year (Australian Institute of Health and Welfare (AIWH), Australia's Health, 2002, Canberra: MW.

A genetic basis for the etiology of obesity is indicated inter alia from studies in twins, adoption studies and population-based analyses which suggest that genetic effects account for 25-80% of the variation in body weight in the general population (Bouchard, 1994, supra, Kopelman et al., Int. J Obesity 18: 188-191, 1994, Ravussin, Metabolism 44(3): 12-14, 1995). It is considered that genes determine the possible range of body weight in an individual and then the environment influences the point within this range where the individual is located at any given time (Bouchard, 1994, supra). However, despite numerous studies into genes thought to be involved in the pathogenesis of obesity, there have been surprisingly few significant findings in this area. In addition, genome-wide scans in various population groups have not produced definitive evidence of the chromosomal regions having a major effect on obesity.

The melanocortin system is one of the critical pathways in the control of human obesity. This system is distributed throughout mammals and consists of: 1) the pro-opiomelanocortin (POMC) precursor hormone which is proteolytically cleaved into several agonist peptides; 2) the melanocortin receptor antagonists agouti and agouti-related peptide (AgRP); and 3) the five seven transmembrane G-protein coupled melanocortin receptors.

The POMC gene encodes a polypeptide precursor and is 7,666 base pairs in length (Bertagna, Endocrinol Metab Clin North Am 23 (3): 467-485, 1994). POMC is proteolytically cleaved into a range of bioactive peptides including ACTH, β-endorphin and most important in the regulation of food intake, α-, β- and γ-melanocyte stimulating hormones (Pritchard et al., J Endocrinol 172 (3): 411-421, 2002). POMC-deficient mice are hyperphagic on normal or high fat chow, and are obese (Yaswen et al., Nat Med 5 (9): 1066-1070, 1999, Krude and Gruters, Trends Endocrinol Metab 11 (1): 15-22, 2000). Human subjects with POMC gene defects have distinctive red hair, adrenal insufficiency and develop severe early-onset obesity (Krude and Gruters, 2000, supra). The obesity in these subjects is reversed when treated by administration of an α-MSH analog (Yaswen et al., 1999, supra). POMC is, therefore, necessary for normal energy balance regulation.

The biological effects of POMC and its cleavage products are thought to be mediated by the 7 transmembrane G-protein coupled melanocortin receptors of which there are 5 known. The basis of the role of POMC in the regulation of energy balance is largely due to the interactions between POMC-derived peptides, particularly α-MSH, and the MC3R and MC4R. Several lines of evidence implicate the MC4R as a controller of feeding behaviour. MC4R-knockout mice have a maturity-onset obesity syndrome associated with hyperphagia, hyperinsulinemia, and hyperglycemia (Huszar et al., Cell 88 (1): 131-141, 1997). This obesity syndrome is very similar to that of A^{y} mice whose obesity occurs as the result of chronic overexpression of agouti protein, a potent antagonist of the MC4R (Yang et al., Mol Endocrinol 11 (3): 274-280, 1997), which competitively inhibits α-MSH binding.

MC4R agonists and antagonists can cause changes in food intake in rodents without any adverse consequences (Fan et al., Nature 385 (6612): 165-168, 1997, Kask and Schioth, Brain Res 887 (2): 460-464, 2000). ICV infusion of MT II, an α-MSH analog and high affinity agonist of MC3R and MC4R, can decrease food intake in mice (Murphy et al., Neuropeptides 32 (6): 491-497, 1998). Administration of SHU9119, an antagonist of the MC4R, increases food intake (Fan et al., 1997, supra). MC4R is further implicated in the regulation of energy balance as mutations in the MC4R gene have been recorded in many obese human subjects. (Vaisse et al., Nat Genet 20 (2): 113-114, 1998, Yeo et al., Nat Genet 20 (2): 111-112, 1998, Hinney et al., J Clin Endocrinol Metab 84 (4): 483-1486, 1999, Farooqi et al., J Clin Invest 106 (2): 271-279, 2000, Branson et al., N Engl J Med 348 (12): 1096-1103, 2003, Farooqi et al., N Engl J Med 348 (12): 1085-1095, 2003, Lubrano-Berthelier et al., Ann N Y Acad Sci 994: 49-57, 2003, Yeo et al., Hum Mol Genet 12 (5): 561-574, 2003). Mutations in the MC4R gene are the most common monogenic form of obesity in humans, accounting for up to 5% of human obesity (Vaisse et al., 2000, supra, Branson et al., 2003, supra), and are therefore relevant major gene effects for obesity (Hinney et al., J Clin Endocrinol Metab 88 (9): 4258-4267, 2003). The functional effects of all of these mutations have yet to be determined, however poor cell surface levels of the mutant receptors are hypothesized to be the mechanism responsible for the development of obesity (Ho and MacKenzie, J Biol Chem 274 (50): 35816-35822, 1999, Nijenhuis et al., J Biol Chem 278 (25): 22939-22945, 2003, VanLeeuwen et al., J Biol Chem 278 (18): 15935-15940, 2003). Many of these mutations are also found to be heterozygous. Thus haploinsufficiency of the MC4R may lead to decreased cell surface expression, decreased response to α-MSH signalling, and subsequent obesity.

The MC3R also has an important but much less defined role in energy balance homeostasis. MC3R knockout mice have a 50-60% increase in fat mass at 4-6 months of age, decreased lean mass, increased feed efficiency, but are not hyperphagic and have normal metabolic rates (Butler et al., Endocrinology 141 (9): 3518-3521, 2000, Chen et al., Nat Genet 26 (1): 97-102, 2000). Interestingly, the MC3R, unlike the MC4R, is co-expressed with POMC in ARC neurons (Jegou et al., J Neuroendocrinol 12 (6): 501-505, 2000), suggesting a regulatory feedback mechanism in the hypothalamus.

Mice lacking both the MC3R and MC4R are significantly heavier than MC4R knockout mice alone (Chen et al., 2000, supra). Therefore, the two melanocortin receptor isoforms reduce body weight through distinct and complementary mechanisms. It has been proposed that the MC4R regulates food intake and possibly energy expenditure, whereas the MC3R influences feed efficiency and partitioning of fuel stores into fat (Cummings and Schwartz, Nat Genet 26 (1): 8-9, 2000).

International Patent Application No. PCT/AU02/01405 which is incorporated herein by reference described the identification of AGT-121 (which is referred to herein as “FIT”), and showed that the level of FIT was associated with obesity and diabetes in a test animal.

In accordance with the present invention, FIT provides a target for therapeutic and diagnostic molecules for conditions such as or involving obesity, anorexia, weight maintenance, inflammation and/or metabolic energy levels.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Nucleotide and amino acid sequences are referred to by a sequence identifier number (SEQ ID NO:). The SEQ ID NOs: correspond numerically to the sequence identifiers <400>1, <400>2, etc. A summary of the sequence identifiers is provided at the end of the specification.

The articles “a” and “an” are used herein to refer to one or more than one (i.e. to at least one) of the grammatical object of the article. By way of example, “an element” means one element or more than one element; “an antagonist” or “an agonist” means a single antagonist or agonist or more than one antagonist or agonist, and so on.

The present invention describes the characterisation of FIT and identifies molecules or “ligands” which interact with FIT as well as antagonists and agonists of the FIT-ligand interaction. Preferred ligands include endophilin 3 and endophilin 1, β-arrestin 1 and 2 and the α-1 and α-2 subunits of the AP2 complex as well as homologs, derivatives or mimetics thereof. All such ligands, antagonists and agonists are referred to as “therapeutic” molecules or may act as part of a diagnostic system. In the case of the latter they may also be referred to as “diagnostic molecules”.

It is proposed that elements of an unhealthy state, including the presence or absence of a disorder or symptom associated with obesity, anorexia, weight maintenance, inflammation and/or metabolic energy levels are modulated by FIT or more particularly the interaction between FIT and its ligands. Therefore, it is proposed that antagonists and agonists of FIT-ligand interaction are useful as therapeutic or prophylactic molecules. Alternatively, the interaction between FIT and its ligands itself may be used as the basis of a diagnostic assay to identify agonists or antagonists of the interaction. In yet a further alternative, FIT itself or its ligands may be useful therapeutic or diagnostic molecules.

The present invention contemplates, therefore, a method for the prophylaxis or treatment of an unhealthy state including a state characterized in part by the presence of a symptom associated with a disorder or disease associated with obesity, anorexia, weight maintenance, inflammation and/or metabolic energy levels in a subject, the method comprising the administration of FIT, a FIT ligand or an agonist or antagonist of a FIT-ligand interaction. The agonists or antagonists contemplated in the present invention also include genetic molecules which modulate the expression of genes encoding the FIT ligands or antagonists or agonists of FIT-ligand interaction.

Examples of antagonists contemplated by the present invention include soluble forms or truncated forms of endophilin 3 and endophilin 1, β-arrestin 1 and 2 and the α-1 and α-2 subunits of the AP2 complex or their homologs, derivatives or mimetics or a non-functional form of FIT which nevertheless binds to a FIT ligand. Other antagonists and agonists may target FIT directly, independent of its interaction with its ligand or visa versa. The antagonists and agonists may be protenaceous, non-protenaceous, small chemical molecules or genetic molecules.

The present invention further contemplates a method for assessing the presence or absence of obesity, anorexia, problems associated with weight maintenance, inflammation and/or abnormal metabolic energy levels or a pre-disposition for development of same, the method comprising determining the level of expression of a nucleic acid molecule which comprises a nucleotide sequence substantially as set forth in SEQ ID NO: 1 or a nucleotide sequence having at least about 40% identity to all or part of SEQ ID NO:1 and/or is capable of hybridizing to SEQ ID NO: 1 or its complementary form under low stringency conditions at a defined temperature or level of expression of a nucleic acid molecule encoding a ligand of the amino acid sequence set forth in SEQ ID NO:2.

In an alternative embodiment, the assay involves measuring or detecting levels of FIT, FIT ligand or modulators of FIT-ligand interaction.

A further aspect of the present invention relates to a composition comprising FIT or a ligand of FIT or an antagonist or agonist of FIT-ligand interaction or their derivatives, homologs, analogs or mimetics together with one or more pharmaceutically acceptable carriers and/or diluents for use in treating conditions associated with of obesity, anorexia, problems with weight maintenance, inflammation and/or abnormal metabolic energy levels.

Reference herein to “FIT” or “AGT-121” includes reference to any homologs, analog, derivative or mimetic thereof from any animal including mammalian or avian species.

A summary of sequence identifiers used throughout the subject specification is provided in Table 1.

TABLE 1
Summary of Sequence Identifiers

| SEQUENCE ID NO. | DESCRIPTION |
|---|---|
| 1 | Nucleotide sequence of AGT-121 or FIT |
| 2 | Amino acid sequence of AGT-121 or FIT |
| 3 | FIT-NP translated sequence in pDBLeu |
| | expression vector |
| 4 | FIT-PR translated sequence in pDBLeu |
| | expression vector |
| 5 | SNP ID 1373910 Forward primer |
| 6 | SNP ID 1373910 Reverse primer |
| 7 | SNP ID 1373910 SNP sequence |
| 8 | SNP ID 1445579 Forward primer |
| 9 | SNP ID 1445579 Reverse primer |
| 10 | SNP ID 1445579 SNP sequence |
| 11 | SNP 1900105 Forward primer |
| 12 | SNP 1900105 Reverse primer |
| 13 | SNP 1900105 SNP sequence |
| 14 | SNP 2146904 Forward primer |
| 15 | SNP 2146904 Reverse primer |
| 16 | SNP 2146904 SNP sequence |
| 17 | SNP 4143026 Forward primer |
| 18 | SNP 4143026 Reverse primer |
| 19 | SNP 4143026 SNP sequence |
| 20 | SNP 604737 Forward primer |
| 21 | SNP 604737 Reverse primer |
| 22 | SNP 604737 SNP sequence |
| 23 | SNP 485521 Forward primer |
| 24 | SNP 485521 Reverse primer |
| 25 | SNP 485521 SNP sequence |
| 26 | SNP1373909 Forward primer |
| 27 | SNP1373909 Reverse primer |
| 28 | SNP1373909 SNP sequence |
| 29 | SNP 4655650 Forward primer |
| 30 | SNP 4655650 Reverse primer |
| 31 | SNP 4655650 SNP sequence |
| 32 | SNP 657808 Forward primer |
| 33 | SNP 657808 Reverse primer |
| 34 | SNP 657808 SNP sequence |
| 35 | SNP 1373911 Forward primer |
| 36 | SNP 1373911 Reverse primer |
| 37 | SNP 1373911 SNP sequence |
| 38 | SNP 2146905 Forward primer |
| 39 | SNP 2146905 Reverse primer |
| 40 | SNP 2146905 SNP sequence |
| 41 | SNP 4655643 Forward primer |
| 42 | SNP 4655643 Reverse primer |
| 43 | SNP 4655643 SNP sequence |
| 44 | SNP1338200 Forward primer |
| 45 | SNP1338200 Reverse primer |
| 46 | SNP1338200 SNP sequence |
| 47 | SNP 502690 Forward primer |
| 48 | SNP 502690 Reverse primer |
| 49 | SNP 502690 SNP sequence |
| 50 | SNP3078564 Forward primer |
| 51 | SNP3078564 Reverse primer |
| 52 | SNP3078564 SNP sequence |
| 53 | SNP 1325267 Forward primer |
| 54 | SNP 1325267 Reverse primer |
| 55 | SNP 1325267 SNP sequence |
| 56 | SNP 1856319 Forward primer |
| 57 | SNP 1856319 Reverse primer |
| 58 | SNP 1856319 SNP sequence |
| 59 | SNP 1325266 Forward primer |
| 60 | SNP 1325266 Reverse primer |
| 61 | SNP 1325266 SNP sequence |
| 62 | SNP 3078564 SNP sequence |
| 63 | FIT-NP sense primer |
| 64 | FIT-PR primer |
| 65 | antisense oligonucleotide |
| 66 | FIT sense primer 5′-tgaaggcttccataggcaaca-3′ |
| 67 | FIT sense primer 5′-tggaacgcctgggtcttg-3′ |

### BRIEF DESCRIPTION OF THE FIGURES

FIG. 1 is a graphical representation of metabolic heat production in Sprague Dawley rats after three days of FIT antisense-treatment compared to jumbled ad libitum fed and jumbled pair fed rats. *p<0.05 pair fed compared to jumbled group, #p<0.05 pair fed compared to FIT antisense-treated group, ̂p<0.05 compared to both jumbled and FIT antisense-treated groups. Data are mean ±SEM.

FIG. 2 is a photographic representation of In situ hybridisation histochemistry of FIT in Psammomys obesus brain sections. Coronal section phosphoimage or P. obesus brain at the region of the anterior hypothalamus (A) and posterior hypothalamus (B) showing FIT mRNA distribution. FIT mRNA distribution at higher magnification in the CA1 region of the hippocampus in light field (C) and dark field (D). Sections of the same region probed with a FIT sense probe in light field (E) and dark field (F) show minimal staining. Cross section of hypothalamic area of lean nGT P. obesus showing POMC mRNA (G); ARC, arcuate nucleus; 3V, third ventricle; VMH, ventromedial hypothalamus. The same section in dark field to visualize FIT mRNA shows a similar expression pattern (H). POMC (1) and increased levels of FIT mRNA (J) are evident in obese D2M P. obesus. High magnification shows co-localisation of FIT mRNA (black dots) with some POMC-containing neurons (large dark cells) (K), and some NPY-containing neurons (L).

### DETAILED DESCRIPTION OF THE PREFERRED EMBODIMENTS

The present invention is predicated in part on the characterization of a molecule associated inter alia with obesity, anorexia, weight maintenance, inflammation and/or abnormal metabolic energy levels. The molecule is referred to herein as AGT121 or FIT. The identification of a nucleic acid molecule encoding FIT, referred to herein as “FIT” is described in PCT/AU02/01405 which is incorporated herein by reference. Although FIT protein is the preferred expression product of FIT, non-protein expression products such as mRNA, RNA including non-coding RNA, introns and exons and RNA/ribosome complexes are also contemplated by the present invention.

The present invention provides, in a particular embodiment, a ligand of FIT.

The nucleotide sequence of FIT is set forth in SEQ ID NO: 1. The amino acid sequence of FIT is set forth in SEQ ID NO:2. The present invention extends to homologs and derivatives having at least 40% similarity to SEQ ID NO:2 or at least 40% identity to SEQ ID NO: 1 or a nucleic acid molecule capable of hybridizing to SEQ ID NO: 1 or its complementary form under low stringey conditions at a particular temperature or range of temperatures.

The term “ligand” means a peptide, polypeptide or protein or chemical molecule which binds, forms a close interaction to or which otherwise associates with FIT. Examples of ligands contemplated by the present invention include cell bound receptors, soluble receptors, intracellular ligands, extracellular ligands and partners in a complex comprising FIT. Non-naturally occurring, chemical molecules including chemical analogs or mimetics of FIT or a FIT ligand are also contemplated. A single ligand may be involved in interaction with the protein or a complex of two or more ligands may be required to from a complex with the subject protein. The term “ligand” also includes binding or interacting partners, cell bound receptors and soluble receptors.

In a preferred embodiment, the ligand for FIT is selected from endophilin 3 and endophilin 1, β-arrestin 1 and 2 and the α-1 and α-2 subunits of the AP2 complex and a homolog, derivative and mimetic thereof. The FIT molecule or its ligand may be from any animal, such as a mammal including a human. Examples of animals include primates such as humans, livestock animals (eg. sheep, cows, horses, donkeys, pigs), laboratory test animals (eg. mice, rats, rabbits, guinea pigs), companion animals and captured wild animals. Particularly preferred mammals include humans.

The present invention provides, therefore, a FIT Ligand, such as selected from endophilin 3 and endophilin 1, β-arrestin 1 and 2 and the α-1 and α-2 subunits of the AP2 complex and a derivative, homolog, analog or mimetic thereof which ligand is capable of interacting with FIT. Preferably, the FIT ligand is a human ligand and FIT is human FIT. Reference to a “FIT ligand” includes polymorphic variants thereof and mutants, derivatives, mimetics and homologs.

The present invention is directed therefore, to a ligand capable of interacting with a protein which comprises the amino acid sequence substantially as set forth in SEQ ID NO:2 or an amino acid sequence having at least 40% similarity thereto after optimal alignment or an amino acid sequence encoded by SEQ ID NO:1 or a nucleotide sequence having at least 40% identity to Seq ID NO:1 or a nucleotide sequence capable of hybridizing to SEQ ID NO:1 or its complementary form under low stringency conditions at a defined temperature and wherein said protein is produced in larger amounts in hypothalamus tissue of obese animals compared to lean animals.

The terms “lean” and “obese” are used in their most general sense but should be considered relative to the standard criteria for determining obesity. Generally, for human subjects the definition of obesity is BMI>30 (Risk Factor Prevalence Study Management Committee. Risk Factor Prevalence Study: Survey No. 3:1989. Canberra: National Heart Foundation of Australia and Australian Institute of Health, 1990; Waters and Bennett, Risk Factors for Cardiovascular Disease: A Summary of Australian data. Canberra: Australian Institute of Health and Welfare, 1995).

Conveniently, an animal model was employed to study the effects of obese and lean animals. In particular, PCT/AU02/01405 exemplified differentially expressed genes using the Psammomys obesus (the Israeli sand rat) animal model of dietary-induced obesity and NIDDM. In its natural desert habitat, an active lifestyle and saltbush diet ensure that they remain lean and normoglycemic (Shafrir and Gutman, 1993, supra). However, in a laboratory setting on a diet of ad libitum chow (on which many other animal species remain healthy), a range of pathophysiological responses are seen (Barnett et al, Diabetologia 37: 671-676, 1994a, Barnett et al., Int. J Obesity 18: 789-794, 1994b, Barnett et al., Diabete Nutr Metab 8: 42-47, 1995). By the age of 16 weeks, more than half of the animals become obese and approximately one third develop NIDDM. Only hyperphagic animals go on to develop hyperglycemia, highlighting the importance of excessive energy intake in the pathophysiology of obesity and NIDDM in Psammomys obesus (Collier et al., Ann New York Acad Sci 827: 50-63, 1997a, Walder et al., Obesity Res 5: 193-200, 1997a). Other phenotypes found include hyperinsulinemia, dyslipidemia and impaired glucose tolerance (Collier et al., 1997a, supra, Collier et al., Exp Clin Endocrinol Diabetes 105: 36-37, 1997b). Psammomys obesus exhibit a range of bodyweight and blood glucose and insulin levels which forms a continuous curve that closely resembles the patterns found in human populations, including the inverted U-shaped relationship between blood glucose and insulin levels known as “Starling's curve of the pancreas” (Barnett et al., 1994a, supra). It is the heterogeneity of the phenotypic response of Psammomys obesus which make it an ideal model to study the etiology and pathophysiology of obesity and NIDDM.

Psammomys obesus animals are conveniently divided into three groups viz Group A animals which are lean, normoglycemic and normoinsulinemic, Group B animals which are obese, normoglycemic and hyperinuslinemic and Group C animals which are obese, hyperglycemic and hyperinsulinemic.

In another embodiment, the present invention provides an antagonist or agonist of interaction between a ligand and a protein which comprises the amino acid sequence substantially as set forth in SEQ ID NO:2 or an amino acid sequence having at least 40% similarity thereto after optimal alignment or an amino acid sequence encoded by SEQ ID NO:1 or a nucleotide sequence having at least 40% identity to SEQ ID NO:1 or a nucleotide sequence capable of hybridizing to SEQ ID NO:1 or its complementary form under low stringency conditions at a defined temperature and wherein said protein is produced in larger amounts in hypothalamus tissue of obese animals compared to lean animals.

Reference herein to similarity or identity is generally at a level of comparison of at least 15 consecutive or substantially consecutive nucleotides or at least 5 consecutive or substantially consecutive amino acid residues. Preferred percentage similarities have at least about 40%, at least about 50%, at least bout 60%, at least about 70%, at least about 80% and at least about 90% or above. Examples include 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and 100%.

The term “similarity” as used herein includes exact identity between compared sequences at the nucleotide or amino acid level. Where there is non-identity at the nucleotide level, “similarity” includes differences between sequences which result in different amino acids that are nevertheless related to each other at the structural, functional, biochemical and/or conformational levels. Where there is non-identity at the amino acid level, “similarity” includes amino acids that are nevertheless related to each other at the structural, functional, biochemical and/or conformational levels. In a particularly preferred embodiment, nucleotide and sequence comparisons are made at the level of identity rather than similarity.

Terms used to describe sequence relationships between two or more polynucleotides or polypeptides include “reference sequence”, “comparison window”, “sequence similarity”, “sequence identity”, “percentage of sequence similarity”, “percentage of sequence identity”, “substantially similar” and “substantial identity”. A “reference sequence” is at least 12 but frequently 15 to 18 and often at least 25 or above, such as 30 monomer units, inclusive of nucleotides and amino acid residues, in length, examples include 12, 13, 14, 15, 16, 17, 18, 19, 20, 21, 22, 23, 24 and 25. Because two polynucleotides may each comprise (1) a sequence (i.e. only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of typically 12 contiguous residues that is compared to a reference sequence. The comparison window may comprise additions or deletions (i.e. gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e. resulting in the highest percentage homology over the comparison window) generated by any of the various methods selected. Reference also may be made to the BLAST family of programs as for example disclosed by Altschul et al. (Nucl. Acids Res. 25: 3389, 1997). A detailed discussion of sequence analysis can be found in Unit 19.3 of Ausubel et al. (“Current Protocols in Molecular Biology” John Wiley & Sons Inc, 1994-1998, Chapter 15).

The terms “sequence similarity” and “sequence identity” as used herein refers to the extent that sequences are identical or functionally or structurally similar on a nucleotide-by-nucleotide basis or an amino acid-by-amino acid basis over a window of comparison.

Thus, a “percentage of sequence identity”, for example, is calculated by comparing two optimally aligned sequences over the window of comparison, determining the number of positions at which the identical nucleic acid base (e.g. A, T, C, G, I) or the identical amino acid residue (e.g. Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software. Similar comments apply in relation to sequence similarity.

Reference herein to a low stringency includes and encompasses from at least about 0 to at least about 15% v/v formamide and from at least about 1 M to at least about 2 M salt for hybridization, and at least about 1 M to at least about 2 M salt for washing conditions. Generally, low stringency is at from about 25-30° C. to about 42° C., such as 25, 26, 27, 28, 29, 30, 31, 32, 33, 34, 35, 36, 37, 38, 39, 40, 41 and 42° C. The temperature may be altered and higher temperatures used to replace formamide and/or to give alternative stringency conditions. Alternative stringency conditions may be applied where necessary, such as medium stringency, which includes and encompasses from at least about 16% v/v to at least about 30% v/v formamide, such as 16, 17, 18, 19, 20, 21, 22, 23, 24, 25, 26, 27, 28, 29 and 30% and from at least about 0.5 M to at least about 0.9 M salt, such as 0.5, 0.6, 0.7, 0.8 and 0.9 M for hybridization, and at least about 0.5 M to at least about 0.9 M salt, such as 0.5, 0.6, 0.7, 0.8 and 0.9 M for washing conditions, or high stringency, which includes and encompasses from at least about 31% v/v to at least about 50% v/v formamide, such as 31, 32, 33, 34, 35, 36, 37, 38, 39, 40, 41, 42, 43, 44, 45, 46, 47, 48, 49 and 50% v/v formamide and from at least about 0.01 M to at least about 0.15 M salt, such as 0.01, 0.02, 0.03, 0.04, 0.05, 0.06, 0.07, 0.08, 0.09, 0.10, 0.11, 0.12, 0.13, 0.14 and 0.15 M for hybridization, and at least about 0.01 M to at least about 0.15 M salt, such as 0.01, 0.02, 0.03, 0.04, 0.05, 0.06, 0.07, 0.08, 0.09, 0.10, 0.11, 0.12, 0.13, 0.14 and 0.15 M for washing conditions. In general, washing is carried out Tₘ=69.3+0.41 (G+C) % (Marmur and Doty, J. Mol. Biol. 5: 109, 1962). However, the Tₘ of a duplex DNA decreases by 1° C. with every increase of 1% in the number of mismatch base pairs (Bonner and Laskey, Eur. J. Biochem. 46: 83, 1974). Formamide is optional in these hybridization conditions. Accordingly, particularly preferred levels of stringency are defined as follows: low stringency is 6×SSC buffer, 0.1% w/v SDS at 25-42° C.; a moderate stringency is 2×SSC buffer, 0.1% w/v SDS at a temperature in the range 20° C. to 65° C.; high stringency is 0.1×SSC buffer, 0.1% w/v SDS at a temperature of at least 65° C.

The present invention extends to a protein form of a FIT ligand including genetic material encoding a FIT ligand. The nucleotide sequence or amino acid sequence of a FIT ligand of the present invention may correspond to exactly the same sequence of the naturally occurring FIT ligand or its gene (or corresponding cDNA) or may carry one or more nucleotide or amino acid substitutions, additions and/or deletions.

As indicated above, preferred ligands are endophilin 3 and endophilin 1, β-arrestin 1 and 2 and the α-1 and α-2 subunits of the AP2 complex or a homolog, derivative or mimetic thereof.

However, any number of approaches may be employed to identify other FIT ligands. In one particularly useful method, a yeast two-hybrid system is employed. The yeast two-hybrid system is an in vivo genetic technique that can be utilized for the identification of protein:protein interactions. The essence of the two-hybrid system is that interaction between two proteins (in this case FIT and a ligand) can be identified by reconstituting active transcription factor dimers. In yeast, these dimers are formed between two fusion proteins, one of which contains a DNA binding (DB) domain fused to the first protein of interest (eg. FIT) and the other, an activation domain (AD) fused to a second protein (eg. FIT ligand). Interaction between DB-FIT and AD-Ligand forms a functional transcription factor that activates chromosomally integrated-reporter genes driven by promoters containing the relevant DB binding sites. When a selectable marker such as HIS3 is used as a reporter gene, two-hybrid dependent transcription activation can be monitored by growth on plates lacking histidine. This technique can, therefore, be applied to test whether two known proteins interact or to detect an unknown protein, encoded by a cDNA library, that interacts with a protein of interest.

Accordingly, another aspect of the present invention contemplates a method of identifying a ligand of the protein FIT or its derivatives, said method comprising introducing a first genetic construct in a yeast strain, said genetic construct comprising a nucleotide sequence encoding all or part of FIT fused to a nucleotide sequence encoding one of a DNA binding (DB) domain or an activation domain (AD) and introducing a second genetic construct into said yeast comprising a cDNA, said second genetic construct comprising elements of a cDNA library fused to a nucleotide sequence encoding the other of a DB domain or AD domain and selecting yeast cells which comprise both genetic constructs and in which a reporter gene has been subjected to two-hybrid dependent transcription.

According to this embodiment, if the cDNA from the cDNA library encodes a binding partner for FIT, then a dimer forms and the DB and AD domains permit transcription of the reporter gene.

In one embodiment, the yeast reporter gene is HIS3 although any other reporter gene may be employed. Preferably, the reporter gene provides a selectable marker.

A homolog of a human FIT ligand is considered to be a homolog or like molecule from another animal species. The present invention extends to the homolog of a FIT ligand selected from endophilin 3 and endophilin 1, β-arrestin 1 and 2 and the α-1 and α-2 subunits of the AP2 complex genes, as determined by nucleotide sequence and/or function and/or amino acid sequence, from non-human primates, livestock animals (e.g. cows, sheep, pigs, horses, donkeys, laboratory test animals (e.g. mice, guinea pigs, hamsters, rabbits), companion animals (e.g. cats, dogs) and captured wild animals (e.g. rodents, foxes, deer, kangaroo).

The present invention extends to the interaction between human FIT and human FIT ligand (homologous system) and human FIT and non-human FIT ligand (heterologous system). In animal systems, a human FIT ligand may be used for non-human FIT.

Apart from the yeast two-hybrid method, the FIT ligands of the present invention may also be identifiable by a number of other means. In one method, FIT or a ligand binding portion thereof is labeled with a reporter molecule and used to screen cells, cell lysate and biological fluid (including blood, serum, lymph fluid) for binding to ligand. For cloning of a FIT ligand, a cDNA library is conveniently prepared and expressed in a suitable cell such as CHO cells and the presence of FIT ligand is then determined by, for example, FIT or a ligand binding portion thereof labeled with a reporter molecule.

Derivatives of a FIT ligand contemplated herein include derivatives of nucleic acid molecules encoding FIT ligand for example, oligonucleotides, PCR primers, antisense molecules, molecules suitable for use in co-suppression and fusion nucleic acid molecules. Ribozymes, DNA enzymes and RNAi are also contemplated by the present invention directed to FIT ligand DNA or mRNA.

Reference herein to a FIT ligand molecule includes reference to isolated or purified naturally occurring FIT ligand as well as any derivatives, homologs, analogs and mimetics thereof. Derivatives include parts, fragments and portions of the FIT ligand as well as single and multiple amino acid substitutions, deletions and/or additions to the FIT partner.

Other derivatives of FIT ligands include chemical analogs. Analogs of a FIT ligand contemplated herein include, but are not limited to, modifications to side chains, incorporation of unnatural amino acids and/or their derivatives during peptide, polypeptide or protein synthesis and the use of crosslinkers and other methods which impose conformational constraints on the proteinaceous molecule or their analogs.

Examples of side chain modifications contemplated by the present invention include those listed in International Patent Application No. PCT/AU98/00902 [WO 99/23217] or U.S. Pat. No. 6,436,670 and which is incorporated herein by reference including incorporation of unnatural amino acids.

The identification of a FIT ligand or an antagonist or agonist of FIT-ligand interaction or of FIT itself permits the development of a range of therapeutic or prophylactic molecules capable of modulating expression of FIT or of a FIT ligand. Modulators contemplated by the present invention includes agonists and antagonists of a FIT ligand expression. Antagonists of a FIT ligand gene expression include antisense molecules, ribozymes and co-suppression molecules including RNAi and siRNA. Agonists include molecules which increase promoter activity or which interfere with negative regulatory mechanisms. Antagonists of a FIT ligand include antibodies and inhibitor peptide fragments as well as small chemical molecule inhibitors. All such molecules may first need to be modified to enable such molecules to penetrate cell membranes. Alternatively, viral agents may be employed to introduce genetic elements to modulate expression of a FIT ligand.

The present invention contemplates, therefore, a method for modulating expression of genetic material encoding a FIT ligand, such as endophilin 3 and endophilin 1, β-arrestin 1 and 2 and the α-1 and α-2 subunits of the AP2 complex or homologs or derivatives thereof, in a mammal, said method comprising contacting the FIT ligand gene the material with an effective amount of a modulator of the expression of the FIT ligand genetic material for a time and under conditions sufficient to up-regulate or down-regulate or otherwise modulate expression of the FIT ligand genetic material. For example, a nucleic acid molecule encoding a FIT ligand, such as one selected from endophilin 3 and endophilin 1, β-arrestin 1 and 2 and the α-1 and α-2 subunits of the AP2 complex or a derivative or homolog thereof may be introduced into a cell.

Another aspect of the present invention contemplates a method of modulating activity of FIT in a mammal, said method comprising administering to said mammal a modulating effective amount of a soluble FIT ligand or a derivative thereof or an antagonist or agonist of FIT-ligand interaction for a time and under conditions sufficient to increase or decrease FIT activity or levels. The derivative of the FIT ligand may be a proteinaceous molecule or a chemical entity such as a product identified from a natural product library or chemical library. Alternatively, derivatives of FIT which are non-functional yet bind to the FIT ligand may also be effective.

One convenient means of screening for antagonists of a FIT ligand or FIT-ligand interaction when in the form of a receptor is to incubate a cell carrying a FIT ligand in the form of a receptor with FIT with or without a potential antagonist and screening for a differential effect when the antagonist is applied. Again, the effect may be gene expression, signal transduction and/or phenotypic changes.

Modulating levels of expression of a FIT ligand or the activity of a FIT ligand or FIT-like interaction is important in the treatment of a range of conditions such as obesity, anorexia, weight maintenance, inflammation and/or metabolic energy levels. It may also be useful in the agricultural industry to assist in the generation of leaner animals, or where required, more obese animals. As indicated above, animals contemplated by the present invention include but are not limited to humans, primates, livestock animals (e.g. pigs, sheep, cows, horses, donkeys), laboratory test animals (e.g. mice, rats, guinea pigs, hamsters, rabbits), companion animals (e.g. dogs, cats) and captured wild animals (e.g. foxes, kangaroos, deer). A particularly preferred host is a human, primate or livestock animal. The present invention further extends to non-mammalian animals such as avian species including poultry birds and game birds.

Accordingly, another aspect of the present invention relates to a method of treating a mammal suffering from a condition characterized by one or more symptoms of an unhealthy state, including the presence or absence of a disorder associated with obesity, anorexia, weight maintenance, inflammation, diabetes, and/or metabolic energy levels comprising administering to said mammal an effective amount of an agent for a time and under conditions sufficient to modulate the activity of FIT or the interaction between FIT and a FIT ligand.

The present invention further contemplates in one embodiment a composition comprising a FIT ligand or a soluble form of a FIT ligand, such as endophilin 3 and endophilin 1, β-arrestin 1 and 2 and the α-1 and α-2 subunits of the AP2 complex or a modulator of a gene expression of a FIT ligand, or an antagonist or agonist of FIT-ligand interaction and one or more pharmaceutically acceptable carriers and/or diluents. One such antagonist includes non-functional FIT derivatives which bind to a FIT ligand.

An “effective amount” means an amount necessary at least partly to attain the desired physiological response, or to delay the onset or inhibit progression or halt altogether, the onset or progression of a particular condition of the individual to be treated, the taxonomic group of the individual to be treated, the degree of protection desired, the formulation of the vaccine, the assessment of the medical situation, and other relevant factors. It is expected that the amount will fall in a relatively broad range that can be determined through routine trials.

In accordance with these methods, therapeutic or prophylactic molecules may be co-administered with one or more other compounds or other molecules. Such molecules include FIT, FIT ligands or modulators of FIT-ligand interaction as well as modulators of expression of genetic molecules encoding FIT or FIT ligands. By “co-administered” is meant simultaneous administration in the same formulation or in two different formulations via the same or different routes or sequential administration by the same or different routes. By “sequential” administration is meant a time difference of from seconds, minutes, hours or days between the administration of the two types of molecules. These molecules may be administered in any order.

The terms “treating” and “treatment” as used herein refer to a reduction in the severity and/or frequency of symptoms associated with inter alia obesity, anorexia, problems with weight maintenance, inflammation and/or abnormal metabolic energy levels, elimination of symptoms and/or the underlying cause, prevention of the occurrence of symptoms of disease and/or the underlying cause and improvement or remediation of damage.

“Treating” a subject may involve prevention of the disorder or disease condition or adverse physiological event in a susceptible individual as well as treatment of a clinically symptomatic individual by inhibiting a disease or disorder. Generally, such conditions involve, weakness (which may be intermittent), neuropathic pain, absent reflexes, gastrointestinal problem (gastroesophogeal reflux, delayed gastric emptying, constipation, pseudo-obstruction), fainting, absent or excessive sweating resulting in temperature regulation problems weakness, hypotonia, cramping, muscle pain, proximal renal tubular wasting resulting in loss of protein, magnesium, phosphorous, calcium and other electrolytes, cardiac conduction defects (heart blocks) and cardiomyopathy, hypoglycemia (low blood sugar) and liver failure, visual loss and blindness, hearing loss and deafness, diabetes and exocrine pancreatic failure (inability to make digestive enzymes), mitochondrial dysfunction, including failure to gain weight, short statue, fatigue and respiratory problems as well as a range of inflammatory conditions.

Examples of inflammatory disease conditions contemplated by the present invention include but are not limited to those diseases and disorders which result in a response of redness, swelling, pain, and a feeling of heat in certain areas that is meant to protect tissues affected by injury or disease. Inflammatory diseases which can be treated using the methods of the present invention, include, without being limited to, acne, angina, arthritis, aspiration pneumonia, disease, empyema, gastroenteritis, inflammation, intestinal flu, NEC, necrotizing enterocolitis, pelvic inflammatory disease, pharyngitis, PID, pleurisy, raw throat, redness, rubor, sore throat, stomach flu and urinary tract infections, Chronic Inflammatory Demyelinating Polyneuropathy, Chronic Inflammatory Demyelinating Polyradiculoneuropathy, Chronic Inflammatory Demyelinating Polyneuropathy, Chronic Inflammatory Demyelinating Polyradiculoneuropathy.

For brevity, all such components of a composition are referred to as “active components”.

The compositions of active components in a form suitable for injectable use include sterile aqueous solutions (where water soluble) and sterile powders for the extemporaneous preparation of sterile injectable solutions. In all cases, the form must be sterile and must be fluid to the extent that easy syringability exists. It must be stable under the conditions of manufacture and storage and must be preserved against the contaminating action of microorganisms such as bacteria and fungi.

The carrier can be a solvent or other medium containing, for example, water, ethanol, polyol (for example, glycerol, propylene glycol and liquid polyethylene glycol, and the like), suitable mixtures thereof, and vegetable oils.

The preventions of the action of microorganisms can be brought about by various antibacterial and antifungal agents, for example, parabens, chlorobutanol, phenol, sorbic acid, thirmerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active components in the required amount in the appropriate solvent with optionally other ingredients, as required, followed by sterilization by, for example, filter sterilization, irradiation or other convenient means. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the active components are suitably protected they may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions is such that a suitable dosage will be obtained. Preferred compositions or preparations according to the present invention are prepared so that an oral dosage unit form contains between about 0.1 μg and 2000 mg of active compound.

The tablets, troches, pills, capsules and the like may also contain the following: A binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such a sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound may be incorporated into sustained-release preparations and formulations.

Pharmaceutically acceptable carriers and/or diluents include any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the novel dosage unit forms of the invention are dictated by and directly dependent on (a) the unique characteristics of the active material and the particular therapeutic effect to be achieved, and (b) the limitations inherent in the art of compounding such an active material for the treatment of disease in living subjects having a diseased condition in which bodily health is impaired as herein disclosed in detail.

The principal active component may be compounded for convenient and effective administration in sufficient amounts with a suitable pharmaceutically acceptable carrier in dosage unit form. A unit dosage form can, for example, contain the principal active component in amounts ranging from 0.5 μg to about 2000 mg. Expressed in proportions, the active compound is generally present in from about 0.5 μg to about 2000 mg/ml of carrier. In the case of compositions containing supplementary active ingredients, the dosages are determined by reference to the usual dose and manner of administration of the said ingredients.

In general terms, effective amounts of the active agents will range from 0.01 ng/kg/body weight to above 10,000 mg/kg/body weight. Alternative amounts range from 0.1 ng/kg/body weight is above 1000 mg/kg/body weight. The therapeutic molecules may be administered per minute, hour, day, week, month or year depending on the condition being treated. The route of administration may vary and includes intravenous, intraperitoneal, subcutaneous, intramuscular, intranasal, via suppository, via infusion, via drip, orally or via other convenient means.

The pharmaceutical composition may also comprise genetic molecules such as a vector capable of transfecting target cells where the vector carries a nucleic acid molecule capable of modulating expression of genetic molecules encoding FIT or FIT ligand or antagonist or agonist of FIT-ligand interaction. The vector may, for example, be a viral vector.

Still another aspect of the present invention is directed to antibodies to FIT or FIT ligand and their derivatives and homologs. Such antibodies may be monoclonal or polyclonal and may be selected from naturally occurring antibodies to FIT or may be specifically raised to FIT or derivatives or homologs thereof. In the case of the latter, FIT or their derivatives or homologs may first need to be associated with a carrier molecule. The antibodies and/or recombinant FIT or their derivatives of the present invention are particularly useful as therapeutic or diagnostic agents and in particular as antagonists of FIT-ligand interaction.

The use of monoclonal antibodies in an immunoassay is particularly preferred because of the ability to produce them in large quantities and the homogeneity of the product. The preparation of hybridoma cell lines for monoclonal antibody production derived by fusing an immortal cell line and lymphocytes sensitized against the immunogenic preparation can be done by techniques which are well known to those who are skilled in the art.

The present invention is further described by the following non-limiting Examples.

### Example 1

### FIT Expression is Increased with Fasting

The effects of fasting on hypothalamic FIT gene expression in Sprague Dawley rats was tested. Semi-quantitative RT-PCR was used to measure FIT gene expression using the primers given in Table 1 above.

TABLE 3
Hypothalamic FIT gene expression in ad libitum fed, 24 hr fasted
and 48 hr fasted Sprague Dawley rats. Data are mean ± SEM

| Group | FIT gene expression (arbitrary units) |
|---|---|
| Fed | 10.1 ± 0.7 |
| 24 hr fasted | 11.7 ± 1.0 |
| 48 hr fasted | 14.7 ± 1.4^{a} |

^{a}p = 0.012 compared to the fed group.

In order to investigate the regulation of FIT mRNA by nutritional status, Sprague Dawley rats were fasted for 24 hr and 48 hr. FIT gene expression was observed to be increased in the hypothalamus of rats fasted for 48 hr compared to fed rats. This suggests that FIT may function as an orexigenic neuroprotein and further implicates FIT in the development or regulation of obesity.

### Example 2

### Suppression of FIT Reduces Food Intake

To further investigate the orexigenic properties of FIT intracerebroventricular (ICV) antisense suppression of FIT was performed. This involved the infusion of FIT antisense oligonucleotides (ODNs) directly into the lateral ventricle space of the brain to suppress endogenous FIT mRNA levels. The effects of this treatment on food intake and body weight were measured and compared to control animals infused with a jumbled sequence ODN, or saline alone. 18 week old, male, lean, normal glucose tolerant (nGT) P. obesus animals were utilised for this study and infused with either saline, FIT antisense ODN, or jumbled ODN at 24 μg/day for four days. The sequence of the FIT antisense ODN was 5′-mu*mg*mg*mc*ma*g*a*a*t*t*g*c*a*mu*mu*mc*mc*mu*-3′, and the jumbled ODN 5′-mc*mg*mc*ma*mc*t*t*a*g*c*t*a*c*mu*mu*mg*mc*mu-3′, where m indicates the presence of a 2′O-Methyl-modified base, and * indicates a phosphorothioate linkage.

TABLE 4
Cumulative food intake of Psammomys obesus intracerebroventricularly
infused with FIT antisense oligonucleotide, jumbled sequence
oligonucleotide, or saline.

| | Day 1 Food | Day 2 Food | Day 3 Food | Day 4 Food |
|---|---|---|---|---|
| Group | (g) | (g) | (g) | (g) |
| FIT antisense- | 6.5 ± 1.6^{a} | 12.3 ± 2.9^{a} | 18.9 ± 3.9^{a} | 27.4 ± 4.4^{a,b} |
| treated | | | | |
| Jumbled- | 8.9 ± 0.5^{a} | 17.3 ± 1.0^{a} | 27.3 ± 1.5^{a} | 38.7 ± 2.7 |
| treated | | | | |
| Saline-treated | 14.2 ± 1.7 | 24.6 ± 1.8 | 35.0 ± 2.1 | 46.3 ± 3.2 |

Data are mean ± SEM.
^{a}p < 0.033 compared to saline-treated group.
^{b}p = 0.015 compared to jumbled-treated group.

ICV FIT antisense treatment significantly reduced cumulative food intake in Psammomys obesus after four days of treatment compared to jumbled oligonucleotide-infused, and saline-treated control animals. This is strong evidence that FIT promotes positive energy balance by regulating appetite, and shows that agents that decrease or inhibit the action of FIT may be useful to regulate appetite and food intake, and therefore to treat obesity.

TABLE 5
Body weight, and change in body weight, of Psammomys obesus
intracerebroventricularly infused with FIT antisense oligonucleotides, jumbled
sequence oligonucleotides, or saline.

| | Day 0 | Day 1 | | | | | | | |
|---|---|---|---|---|---|---|---|---|---|
| | BW | BW | | Day 2 | | Day 3 | | Day 4 | |
| Group | (g) | (g) | ΔBW(g) | BW(g) | ΔBW(g) | BW(g) | ΔBW(g) | BW (g) | ΔBW(g) |
| FIT | 199 ± 7 | 195 ± 7 | −4.5 ± 0.7^{a} | 194 ± 8 | −5.7 ± 2.1^{a} | 192 ± 7 | −7.7 ± 1.8^{a} | 190 ± 7 | −9.2 ± 1.5^{a,b} |
| antisense- | | | | | | | | | |
| treated | | | | | | | | | |
| Jumbled- | 191 ± 9 | 188 ± 9 | −3.8 ± 0.7 | 188 ± 9 | −3.3 ± 0.8 | 187 ± 9 | −4.0 ± 1.6 | 187 ± 9 | −3.5 ± 1.7 |
| treated | | | | | | | | | |
| Saline- | 191 ± 7 | 190 ± 8 | −1.9 ± 1.2 | 191 ± 8 | −0.9 ± 1.4 | 190 ± 8 | −1.9 ± 1.0 | 190 ± 8 | −1.1 ± 0.6 |
| treated | | | | | | | | | |

BW, body weight;
Δ, change in (compared to day 0).
Data are mean ± SEM.
^{a}p < 0.02 compared to the saline-treated group.
^{b}p = 0.018 compared to the jumbled-treated group.

ICV antisense suppression of FIT also reduced body weight in Psammomys obesus over four days compared to the jumbled oligonucleotide-infused, and saline-treated control animals. Therefore, suppression of FIT in P. obesus inhibited food intake and reduced body weight, providing strong evidence for a role for FIT in the central regulation of energy balance and suggests that agents that decrease or inhibit the action of FIT may be useful as obesity therapeutics.

### Example 3

### FIT Suppression Decreases Appetite and Induces Weight Loss

To confirm the appetite-suppressing and weight-reducing effects of central FIT suppression in an animal model other than P. obesus, the inventors performed the same experiments in Sprague Dawley rats. Male, 12 week old Sprague Dawley rats were infused with either saline, FIT antisense ODN, or jumbled ODN at 24 μg/day for four days. The sequence of the FIT antisense ODN and the jumbled ODN are the same as in Example 2 above.

TABLE 6
Cumulative food intake of Sprague Dawley rats intracerebroventricularly
infused with FIT antisense oligonucleotides, jumbled sequence
oligonucleotides, or saline.

| | Day 1 Food | Day 2 Food | Day 3 Food | Day 4 Food |
|---|---|---|---|---|
| Group | (g) | (g) | (g) | (g) |
| FIT | 8.6 ± 1.6^{a,b} | 23.4 ± 2.6^{a,b} | 38.3 ± 3.6^{a,b} | 55.0 ± 4.9^{a,b} |
| antisense- | | | | |
| treated | | | | |
| Jumbled- | 18.7 ± 2.4 | 44.7 ± 4.1 | 72.6 ± 5.1 | 101.5 ± 6.1 |
| treated | | | | |
| Saline- | 16.8 ± 6.9 | 36.7 ± 3.0 | 62.2 ± 3.7 | 87.1 ± 4.1 |
| treated | | | | |

Data are mean ± SEM.
^{a}p < 0.03 compared to saline-treated group.
^{b}p < 0.004 compared to jumbled-treated group.

ICV antisense suppression of FIT reduced cumulative food intake in Sprague Dawley rats after four days compared to jumbled oligonucleotide-infused, and saline-treated control animals. Therefore, FIT plays a key role in regulating feeding behaviour, and is not limited to regulation of food intake in P. obesus alone.

### Example 4

### Suppression of FIT Reduces Body Weight

ICV antisense suppression of FIT also reduced body weight in Sprague Dawley rats over four days compared to jumbled oligonucleotide-infused, and saline-treated control animals. This loss of body weight was very similar to the degree of weight loss seen in P. obesus, and provides strong evidence of a key role for FIT in the regulation of energy balance and development of obesity. Agents that inhibit or block FIT may be useful in the treatment of obesity.

TABLE 7
Body weight, and change in body weight, of Sprague Dawley rats
intracerebroventricularly infused with FIT antisense oligonucleotides, jumbled
sequence oligonucleotides, or saline.

| | | Day 1 | | | | | | | |
|---|---|---|---|---|---|---|---|---|---|
| | Day 0 | BW | | Day 2 | | Day 3 | | Day 4 | |
| Group | BW(g) | (g) | ΔBW(g) | BW(g) | ΔBW(g) | BW(g) | ΔBW(g) | BW (g) | ΔBW(g) |
| FIT | 444 ± 37 | 425 ± 37 | −19.6 ± 2.9^{b} | 415 ± 37 | −28.8 ± 4.1^{a,b} | 410 ± 38^{a} | −34.4 ± 4.1^{a,b} | 408 ± 37^{a} | −36.6 ± 6.7^{a,b} |
| antisense- | | | | | | | | | |
| treated | | | | | | | | | |
| Jumbled- | 465 ± 24 | 457 ± 24 | −7.8 ± 3.6 | 468 ± 23 | 3.5 ± 3.7 | 473 ± 23 | 6.3 ± 3.5 | 477 ± 22 | 12.0 ± 4.1 |
| treated | | | | | | | | | |
| Saline- | 507 ± 35 | 496 ± 35 | −11.1 ± 3.2 | 497 ± 32 | −9.8 ± 4.4 | 508 ± 31 | 1.8 ± 4.4 | 508 ± 30 | 1.8 ± 5.6 |
| treated | | | | | | | | | |

BW, body weight;
Δ, change in (compared to day 0).
Data are mean ± SEM.
^{a}p < 0.04 compared to saline-treated group.
^{b}p < 0.03 compared to jumbled-treated group.

### Example 5

### FIT Suppression Using ODNs Reduced mRNA Levels in the Hypothalamus

To confirm endogenous suppression of FIT in antisense-treated Sprague Dawley rats, FIT mRNA in the hypothalamus of these animals was measured. Semi-quantitative RT-PCR was used to measure FIT gene expression using the primers provided in Table 1 above.

TABLE 8
FIT gene expression was measured in the hypothalamus of FIT ICV
antisense-treated Sprague Dawley rats.

| Group | FIT gene expression (arbitrary units) |
|---|---|
| FIT antisense-treated | 6.6 ± 1.0^{a} |
| Jumbled-treated | 8.4 ± 0.6 |
| Saline-treated | 10.5 ± 1.1 |

Data are mean ± SEM.
^{a}p = 0.008 compared to saline-treated group.

FIT gene expression was suppressed by 37% compared to saline-treated rats, and by 20% compared to jumbled-treated rats. This indicates that the FIT antisense ODN was actively suppressing mRNA levels in the hypothalamus, and suggests that the inhibition of food intake and reduction in body weight was specifically due to this effect and not to non-specific effects or illness.

### Example 6

### FIT Suppression Results in Weight Loss Due to Decreases in Food Intake and High Energy Expenditure

To investigate the mechanism of weight loss after FIT antisense treatment, a group of Sprague Dawley rats were pair fed to the average daily food intake levels of FIT antisense-treated rats, and indirect calorimetry (24 h) was performed before and after FIT antisense or jumbled ODN ICV infusion.

TABLE 9
Phenotypic and energy expenditure data for jumbled-treated ad libitum fed,
jumbled-treated pair fed, and FIT antisense-treated rats.

| | Day 0 | Day 4 BW | Δ BW | Food | Pre TEE | Post TEE | Δ TEE |
|---|---|---|---|---|---|---|---|
| Group | BW(g) | (g) | (g) | Intake(g) | (kJ/day/kg) | (kJ/day/kg) | (kJ/day/kg) |
| Jumbled | 404 ± 31 | 386 ± 24 | −17.3 ± 8.9 | 81.1 ± 10.3 | 513 ± 62 | 492 ± 61 | −21 ± 17 |
| Jumbled | 357 ± 9 | 317 ± 7^{a} | −39.7 ± 9.9 | 39.1 ± 3.2^{a} | 556 ± 59 | 456 ± 30^{c} | −100 ± 28 |
| pair fed | | | | | | | |
| FIT | 408 ± 13 | 338 ± 12^{a,c} | −70.0 ± 8.6^{a,b} | 36.4 ± 11.2^{a} | 592 ± 30 | 525 ± 22 | −67 ± 25 |
| antisense | | | | | | | |

BW, body weight;
TEE, total energy expenditure;
RQ, respiratory quotient.
Data are mean ± SEM.
^{a}p < 0.05 compared to jumbled-treated ad libitum fed group.
^{b}p < 0.05 compared to jumbled-treated pair fed group.
^{c}p < 0.05 compared to pre data within the same group.

Pair fed rats exhibited greater loss of body weight than that of jumbled ad libitum fed rats, but less than FIT antisense treated rats, even though the pair fed group consumed the same amount of food as FIT antisense-treated rats. This indicates that the weight loss observed after FIT antisense-treatment cannot be explained by the inhibition of food intake alone, and that other mechanisms of weight loss must be occurring in these animals.

Total energy expenditure (TEE) was calculated from measurements of oxygen consumption and carbon dioxide production using indirect calorimetry (Ferrannini, 1988). Due to differences in body weight between the three groups of rats, TEE levels were calculated relative to whole body mass of the animals. It was observed that pair fed rats significantly reduced their TEE, probably as a compensatory mechanism to conserve energy after reduced food intake. FIT antisense-treated rats, however, were unable to significantly reduce their TEE levels. FIT antisense-treated rats therefore have an inappropriately high level of energy expenditure for their body weight. FIT antisense-treated rats exhibit reduced food intake, as well as an inability to reduce TEE, and these two factors contribute to the large reductions in body weight observed in these rats.

### Example 7

### Suppression of FIT is Associated with an Inability to Reduce TEE

To further dissect the nature of the inability to reduce TEE levels after FIT antisense treatment, metabolic heat production was measured by indirect calorimetry.

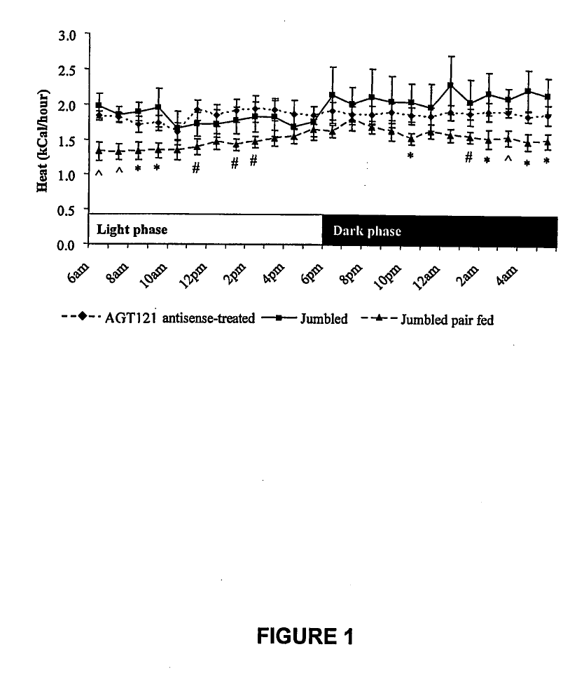

Jumbled-treated pair fed animals showed significantly reduced heat production compared to both jumbled-treated ad libitum fed and FIT antisense-treated rats, particularly throughout the last half of the dark phase and majority of the light phase (refer to FIG. 1). Therefore, an inability to reduce heat production, or uncontrolled thermogenesis, may be a contributor to the high levels of TEE seen in FIT antisense-treated rats.

### Example 7

### FIT Suppression Does Not Impact Levels of Physical Activity

Levels of physical activity also contribute to whole body energy expenditure. Therefore physical activity levels of the rats were measured while in the calorimetry chamber.

TABLE 10
Total activity levels of FIT antisense-treated, jumbled-treated ad
libitum fed, and jumbled-treated pair fed animals.

| | Light Phase | | Dark Phase | |
|---|---|---|---|---|
| Group | 6 am-12 pm | 12 pm-6 pm | 6 pm-12 am | 12 am-6 am |
| Jumbled | 2274 ± 550 | 2783 ± 492 | 5764 ± 1187^{b} | 5345 ± 746^{a,b} |
| Jumbled | 2059 ± 311 | 3349 ± 505 | 5170 ± 1322 | 3081 ± 577 |
| pair fed | | | | |
| FIT | 2052 ± 119 | 2316 ± 240 | 3348 ± 472 | 2391 ± 463 |
| antisense | | | | |

Data is represented as the sum of activity measures and grouped as 6 hour bins.
(mean ± SEM)
^{a}p < 0.023 compared to FIT antisense-treated and jumbled-treated pair fed group, 12 am to 6 pm time bin.
^{b}p < 0.026 compared to light phase activity levels within the jumbled-treated ad libitum fed group.

Jumbled-treated ad libitum fed rats exhibited increased activity in the dark phase compared to the light phase. Pair fed rats and FIT antisense-treated rats did not exhibit a statistically significant increase in physical activity in the dark phase. Differences in physical activity levels do therefore not account for the differences in TEE levels. FIT antisense-treatment therefore affects whole body energy balance by reducing food intake and causing an inappropriately high metabolic rate, rather than affecting physical activity of these rats.

### Example 9

### FIT Regulates Energy Imbalance

To investigate the mechanism by which FIT regulates energy balance, neural proteins that interact with FIT were identified. In order to perform these experiments, two truncated fragments of FIT were constructed to be used as baits in a yeast two-hybrid screen of a human brain cDNA library: one bait consisting of the N-terminal region plus the central proline-rich region, and the other bait consisting of the central proline-rich region alone. The inventors utilised a yeast two-hybrid system which incorporates modifications by Chevray and Nathans (1992), and Vidal et al. (1996) to screen a human brain cDNA library. FIT fragments were PCR amplified, purified and then cloned into an appropriate vector. Sense oligonucleotides for FIT-NP (5′-gtacagtcgactatgatggaaggactgaaaaaacg-3′: SEQ ID NO:72), and FIT-PR (5′-gtacagtcgaccagaccttttcccactg-3′: SEQ ID NO:73), with Sal1 restriction enzyme sites underlined, were used in conjunction with a common antisense oligonucleotide (5′-atagcggccgcggctaagggtgctat-3′: SEQ ID NO:74), with the Not1 restriction enzyme site underlined.

FIT-NP translated sequence in expression vector.

(SEQ ID NO:3)

MMEGLKKRTRKAFGIRKKEKDTDSTGSPDRDGMQPSPHELPYHSKAECAR
EGGKKASKKSNGAPNGFYAEIDWERYNSPELDEEGYSIRPEEPGSTKGKH
FYSSSESEEEEESHKKFNIKIKPLQSKDILKNAATVDELKASIGNIALSP
SPVRKSPRRSPGAIKRNLSSEEVARPRRSTPTPELTSKKPLDDTLALAPL
FGPPLESAFDGHKTEVLLDQPEIWGSGQPVNPSMESPKLARPFPTGTPPP
LPPKTVPATPPRTGSPLTVATGNDQAATEAKIEKPPSISDLDSIFGPVLS
PKSVAVNTEETWVHFSDASPEHVTPELTPREKVVTPPAASDIPADSPTPG
PPGPPGSAGPPGPPGPRNVPSPLNLEEVQKKVAEQTFIKDDYLETLSSPK
ECGLGQRETPPPPPPPTYRTVVSSPGPGSGSGTGTASGASSPARPATPLV
PCSCSTPPPPPPRPPSRPKLPPGKPGVGDVSRPFSPPIHSSSPPPIAPL
FIT-PR translated sequence in expression vector.

(SEQ ID NO:4)

RPFPTGTPPPLPPKTVPATPPRTGSPLTVATGNDQAATEAKIEKPPSISD
LDSIFGPVLSPKSVAVNTEETWVHFSDASPEHVTPELTPREKVVTPPAAS
DIPADSPTPGPPGPPGSAGPPGPPGPRNVPSPLNLEEVQKKVAEQTFIKD
DYLETLSSPKECGLGQRATPPPPPPPTYRTVVSSPGPGSGSGTGTASGAS
SPARPATPLVPCSCSTPPPPPPRPPSRPKLPPGKPGVGDVSRPFSPPIHS
SSPPPIAPL

Human brain cDNA inserts were prepared and cloned into the expression vector according to manufacturer's instructions.

TABLE 11
FIT interacting proteins isolated from a human brain cDNA library
using a yeast two-hybrid screen.

| | Number | |
|---|---|---|
| Bait | of clones | Protein |
| FIT-NP | 7 | SH3GL3; endophilin 3 |
| FIT-NP | 14 | SH3GL2; endophilin 1 |
| FIT-NP | 1 | Adapter-related protein complex 2, α-2 subunit |
| FIT-NP | 8 | Adapter-related protein complex 2 α-1 subunit |
| FIT-NP | 2 | Beta-arrestin 2 |
| FIT-NP | 3 | Polyubiquitin C |
| FIT-NP | 10 | Homo sapiens regulatory factor X, 2 (RFX2) |
| FIT-NP | 1 | Beta-arrestin 1 |
| FIT-NP | 1 | RNA-binding protein EWS |
| FIT-NP | 1 | MAD2 |
| FIT-PR | 12 | SH3GL2; endophilin 1 |
| FIT-PR | 2 | SH3GL3; endophilin 3 |
| FIT-PR | 1 | D. melanogaster transcription factor CG4654-PB |
| FIT-PR | 1 | Homo sapiens regulatory factor X, 2 (RFX2) |

FIT-PR, FIT proline-rich region; FIT-NP, FIT N-terminal plus proline-rich region. The human cDNA clones are listed in order of highest to lowest interacting strength.

FIT was observed to interact very strongly with endophilin 3 and endophilin 1, proteins with known roles in synaptic vesicle recycling and clathrin mediated endocytosis of ligand-bound cell surface receptors. FIT also interacted with β-arrestin 1 and 2, and the α-1 and α-2 subunits of the AP2 complex at slightly lesser strength in yeast. β-arrestins and AP2 have also been shown to be necessary for ligand bound receptor internalisation. These results suggest a potential role for FIT in the regulation of receptor-mediated signalling pathway(s), possibly by regulating receptor internalisation.

### Example 10

### FIT Molecular Pathway and Food Intake

To investigate the molecular pathway via which FIT affect food intake, FIT gene expression was measured in the hypothalamus of several different monogenic mouse models of obesity. FIT gene expression was elevated approximately four fold in the hypothalamus of obese agouti (A^{y}) mice, as well as A^{y} mice that had been dietary energy restricted for twenty days and had a lean phenotype. FIT gene expression was not altered in other genetic models of obesity such as leptin deficient ob/ob mice, MC3R knockout mice and MC4R knockout mice.

TABLE 12
FIT gene expression in the hypothalamus of monogenic mouse models of
obesity.

| | FIT gene |
|---|---|
| Group | expression (arbitrary units) |
| Wild-type mice | 1.4 ± 0.4 |
| Ob/ob mice | 1.3 ± 0.1 |
| Leptin-treated ob/ob mice | 1.3 ± 0.3 |
| Agouti mice | 4.0 ± 0.4^{a} |
| Energy restricted agouti mice | 3.2 ± 0.3^{a} |
| Melanocortin 3 receptor knockout mice | 1.4 ± 0.1 |
| Melanocortin 4 receptor knockout mice | 1.3 ± 0.1 |

Data are mean ± SEM.
^{a}p < 0.004 compared to wild-type mice.

A^{y} mice overexpress the endogenous protein agouti which is an antagonist at MC3 and MC4 receptors. Therefore FIT appears not to be directly regulated by all conditions affecting body weight or adiposity, but rather may be affected by a central pathway known to regulate energy balance, namely agouti-induced obesity and the melanocortin signalling pathway.

Given the previous findings that FIT is involved in the regulation of food intake and body weight, most likely through modulation of clathrin-mediated endocytosis of ligand-bound cell surface receptors, these data suggest that FIT is a major factor in the regulation of cell surface levels of MC3R and MC4R. As such, agents that affect the activity of FIT could alter the melanocortin system to regulate food intake and energy balance.

### Example 11

### FIT and the Melanocortin System

To further analyse the link between FIT and the melanocortin system, the feeding response of Sprague Dawley rats to an ICV injection of SHU9119, a melanocortin receptor antagonist and known feeding stimulator was investigated. After three days of FIT antisense treatment, jumbled oligonucleotide or saline infusion, responses were compared to rats receiving an ICV injection of saline vehicle rather than SHU9119.

TABLE 13
48 hr food intake of Sprague Dawley rats ICV infused with FIT antisense ODN,
jumbled sequence ODN, or saline and injected with either SHU9119 or saline. Data
is represented as food intake in grams relative to the body weight of the animal in
grams.

| | | 48 hr FI pre injection | 48 hr FI post injection | Δ FI |
|---|---|---|---|---|
| Group | n | (g/g BW) | (g/g BW) | (g/g BW) |
| Saline + Vehicle | 3 | 0.13 ± 0.01 | 0.14 ± 0.01 | 0.01 ± 0.01 |
| Saline + SHU9119 | 5 | 0.14 ± 0.01 | 0.16 ± 0.01^{a} | 0.02 ± 0.01 |
| Jumble + Vehicle | 5 | 0.10 ± 0.01 | 0.13 ± 0.01 | 0.03 ± 0.02 |
| Jumble + SHU9119 | 5 | 0.13 ± 0.01 | 0.17 ± 0.01^{a} | 0.04 ± 0.01 |
| FIT antisense + Vehicle | 4 | 0.07 ± 0.01^{b} | 0.09 ± 0.01^{b,d} | 0.02 ± 0.01 |
| FIT antisense + SHU9119 | 4 | 0.07 ± 0.01^{c} | 0.14 ± 0.01^{a} | 0.08 ± 0.01^{c} |

FI, food intake;
BW, body weight.
Data are mean ± SEM.
^{a}p < 0.05 compared to 48 hr pre FI data.
^{b}p < 0.05 compared to saline and jumbled + vehicle groups.
^{c}p < 0.02 compared to saline and jumbled + SHU9119 groups.
^{d}p < 0.05 compared to FIT antisense + SHU9119 group.

FIT antisense-treated rats demonstrated a significantly increased feeding response after SHU9119 injection compared to jumbled-treated or saline-treated rats relative to body weight. Food intake was increased in both of the control groups after SHU9119 injection but the degree of food intake increase was far greater after FIT antisense-treatment. Therefore, suppression of endogenous FIT levels in the hypothalamus potentiated the effect of SHU9119. These data support our contention that FIT regulates the melanocortin system, probably by affecting internalisation and cell-surface levels of MC3R and MC4R in the hypothalamus. Specifically, these results suggest that suppression of FIT using antisense ODN impaired the internalisation and/or recycling of MC3R/NC4R, resulting in increased cell surface levels of one or both of these receptors. Injection of SHU9119 then had a greater effect in these animals due to the increased density of cell surface melanocortin receptors in the hypothalamus.

To further investigate this relationship, antisense suppression of FIT in Sprague Dawley rats was again performed and these animals' responses to administration of the α-MSH analog MT II, a synthetic agonist of the MC4R and known suppressor of feeding. MT II effects on food intake and body weight in saline and jumbled ODN-treated control rats, as well as in FIT antisense-treated rats was measured.

TABLE 14
24 hr food intake of Sprague Dawley rats ICV infused with FIT antisense ODN,
jumbled sequence ODN, or saline and injected with either MT II or saline.

| | | 24 hr FI pre injection | 24 hr FI post injection | Δ FI |
|---|---|---|---|---|
| Group | n | (g/g BW) | (g/g BW) | (g/g BW) |
| Saline + Vehicle | 3 | 0.06 ± 0.01 | 0.08 ± 0.01^{a,d,e} | 0.02 ± 0.01^{b,d,e} |
| Saline + MT II | 3 | 0.07 ± 0.01 | 0.03 ± 0.01^{a} | −0.04 ± 0.01 |
| Jumbled + Vehicle | 5 | 0.06 ± 0.01 | 0.04 ± 0.01 | −0.02 ± 0.01 |
| Jumbled + MT II | 4 | 0.04 ± 0.01^{b} | 0.01 ± 0.01^{a} | −0.03 ± 0.01 |
| FIT antisense + Vehicle | 4 | 0.04 ± 0.01^{d} | 0.03 ± 0.01 | −0.01 ± 0.01^{c} |
| FIT antisense + MT II | 4 | 0.04 ± 0.01^{b} | 0.01 ± 0.01^{a} | −0.04 ± 0.01^{e} |

FI, food intake;
BW, body weight.
Data are mean ± SEM.
^{a}p < 0.022 compared to 24 hr pre FI data.
^{b}p < 0.05 compared to saline + MT II group.
^{c}p < 0.05 compared to saline + vehicle group.
^{d}p < 0.05 compared to jumbled + vehicle group.
^{e}p < 0.05 compared to FIT antisense + vehicle group.

Although not statistically significantly different from the control groups, MT II tended to have a greater inhibitory effect on food intake in rats pre-treated with FIT antisense ODN. These results support our hypothesis that FIT is involved in the regulation of melanocortin signalling.

### Example 12

### FIT SNPs and Obesity

To further investigate the importance of FIT in the development of human obesity, an association study between FIT single nucleotide polymorphisms (SNPs) and obesity phenotypes in a human population was performed. Twenty-eight conserved SNPs were selected for genotyping in FIT from on-line databases. These were genotyped in the founders of the Mauritian extended pedigree collection (n=65). This family based collection was established for the study of inherited factors predisposing to the development of type 2 diabetes, obesity and related disorders associated with the metabolic syndrome. Redundant SNPs were removed and the remaining 19 SNPs were then genotyped in the whole cohort (n=400) by mass spectrometry. Primer sequences and the sequence of the SNPs are shown in the table below.

TABLE 17

SNP identification number, sequences and primer sequences
used for the association study in the Mauritian subjects.

| | | | |
|---|---|---|---|
| SNP ID | | | |
| (rs) | Forward Primer | Reverse Primer | SNP Sequence |

| | | | |
|---|---|---|---|
| 1373910 | gttgttctatcactggcagtc | gagcattgcaaagaggatggg | caaagaggatgggagggttt[a/g]tattaggtcctgtgtg |
| | SEQ ID NO:5 | SEQ ID NO:6 | SEQ ID NO:7 |
| 1445579 | gcaagcaaactgcagcatttc | gatgtgcagccagtgtatgtg | ttctagaacctggttgccaa[g/t]gttttgcaagcagaaatgct |
| | SEQ ID NO:8 | SEQ ID NO:9 | SEQ ID NO:10 |
| 1900105 | gttcttccctctgggtctatc | gaataaggaaaggcctccagc | tgggtctatctcctgctctgtg[c/g]ctttacctctggtcacagg |
| | SEQ ID NO:11 | SEQ ID NO:12 | SEQ ID NO:13 |
| 2146904 | gaactgtcatgcaacctgctg | gctcagatgcaccctgtatat | tgtatatttactgttcatc[a/g]tggaactcgtgccactga |
| | SEQ ID NO:14 | SEQ ID NO:15 | SEQ ID NO:16 |
| 4143026 | gcaaattagcctgccagagag | gtgaagtgaggacaggaaagg | gcaaaattcatgaatttgc[c/t]gctgcttggtaacaccacccc |
| | SEQ ID NO:17 | SEQ ID NO:18 | SEQ ID NO:19 |
| 604737 | ggtgccattcaacaatactac | gggcagttagacacttgagtt | ttttaccattcctgaaatggat[c/t]taatttaaactgtggtatgt |
| | SEQ ID NO:20 | SEQ ID NO:21 | SEQ ID NO:22 |
| 485521 | ggtaaaaagggaaagcaattc | ggagaggggcaagtagttaag | tcaagagtaaagaagatg[a/g]tgaagtcttaactacttgcccc |
| | SEQ ID NO:23 | SEQ ID NO:24 | SEQ ID NO:25 |
| 1373909 | gctcccatcctctttgcaatg | gttctgcttagaaggcttggg | gtgttcatggagataacagc[a/g]aatggtcttccaggaattta |
| | SEQ ID NO:26 | SEQ ID NO:27 | SEQ ID NO:28 |
| 4655650 | gtgcaggcgttttcagttttg | gcagacattaaccccatgaac | cgttttcagttttgaagcatatt[c/t]ataggaggctttaaatca |
| | SEQ ID NO:29 | SEQ ID NO:30 | SEQ ID NO:31 |
| 657808 | gtaaaactctccttctggatc | gacccacaggaatcaaaacgc | aattttagggaaaaaaaagtcc[a/g]ctgtttagatccagaag |
| | SEQ ID NO:32 | SEQ ID NO:33 | SEQ ID NO:34 |
| 1373911 | gccccatttcatttggccaac | gtttggggatgcatctacaag | tcttaaatttacttrgcctta[c/t]gtttagatccaacttggat |
| | SEQ ID NO:35 | SEQ ID NO:36 | SEQ ID NO:37 |
| 2146905 | gtctcacatgcagccacaaag | gtgctccccagaaaattggtc | taattcattcatttgagagac[a/c]ctaaaggaaggaaaattg |
| | SEQ ID NO:38 | SEQ ID N0:39 | (SNP at position 166851) |
| | | | SEQ ID N0:40 |
| 4655643 | ggggtgggtagttttaaatgtc | gactattttccgttactctcc | tacrttttcctataaactc[a/c]tcatgtggagagtaacggaa |
| | SEQ ID NO:41 | SEQ ID NO:42 | SEQ ID NO:43 |
| 1338200 | gatgaactgcagaggcagtac | gttttccaaatgaaaatacag | tgaaaatacagagcgaga[a/c]agctttttttaaaaaaaata |
| | SEQ ID NO:44 | SEQ ID NO:45 | SEQ ID NO:46 |
| 502690 | gccccaagaacctcaggaaat | gtacttttcagagcaaagcac | tttaaataataaaaatgatgt[a/t]tatatgtgtgctttgctctg |
| | SEQ ID NO:47 | SEQ ID NO:48 | SEQ ID NO:49 |
| 3078564 | ggattcagtgtattgacatgg | gtgacaacaccatttctccgg | gtattgacatggattttctctc[deltc]ntttcctctctgtgtttt |
| | SEQ ID NO:50 | SEQ ID NO:51 | SEQ ID NO:52/62 |
| 1325267 | gtgctgaatgacagtttgccc | gatggagcagaagtcttcctg | gtgcagttaaaatatgctga[c/t]gcccctgcatggccagga |
| | SEQ ID NO:53 | SEQ ID NO:54 | SEQ ID NO:55 |
| 1856319 | gccaacttccttttgtagagc | gttagatgtggaaaacttgcc | aatcaaggggaaagaaaa[c/t]ttgaattgctctacaaaag |
| | SEQ ID NO:56 | SEQ ID NO:57 | SEQ ID NO:58 |
| 1325266 | ggggtgttttgtgtctggatg | gcagggaagatgtcacatatc | ggatgcctaaggtgattcca[a/g]gggaggggatggaagata |
| | SEQ ID NO:59 | SEQ ID NO:60 | (SNP at position 160560) |
| | | | SEQ ID NO:61 |

The potential of genetic variants within the FIT gene were examined to determine if they have an effect on a number of phenotypes related to the metabolic syndrome in Mauritian families. Phenotypes included BMI, diabetes affection status, fat mass, waist hip ratio, cholesterol levels, glycosylated hemoglobin, fasting glucose, fasting insulin, and urate levels. Preliminary analysis yielded nominal associations of FIT SNPs with various diabetes and obesity phenotypes.

TABLE 16
Summary of SNPs in FIT sequence in human Mauritian population.

| SNP ID | SNP Position | Phenotype | P-value |
|---|---|---|---|
| rs1856319 | Intron 1 | Cholesterol | 0.0386 |
| rs1373909 | Intron 1 | Cholesterol | 0.0295 |
| rs1373910 | Intron 1 | Fat free mass | 0.0303 |
| rs1900105 | Intron 1 | Fat free mass | 0.0267 |
| rs4655650 | Intron 10 | Diabetes | 0.0479 |
| | | Fat mass | 0.0206 |
| | | Weight | 0.0384 |
| rs1325266 | Intron 15 | Cholesterol | 0.0216 |
| | | Fat mass | 0.0019 |
| rs1325267 | Intron 15 | Cholesterol | 0.0355 |
| | | Waist-hip ratio | 0.0500 |
| rs2146905 | Intron 16 | Cholesterol | 0.0204 |
| | | Fat mass | 0.00057 |
| | | Fat free mass | 0.0324 |
| | | LDL cholesterol | 0.0425 |
| rs604737 | 3′ UTR | Diabetes | 0.0117 |

Examination of this evidence suggests that FIT is involved in aspects of the metabolic syndrome in humans. Both obesity and diabetes show strong associations with FIT variants as does cholesterol metabolism. These results suggest that FIT is involved in the regulation of these processes in human subjects.

### Example 13

### FIT Expression in Brain

The distribution of FIT mRNA throughout P. obesus brain was examined by in situ hybridisation histochemistry.

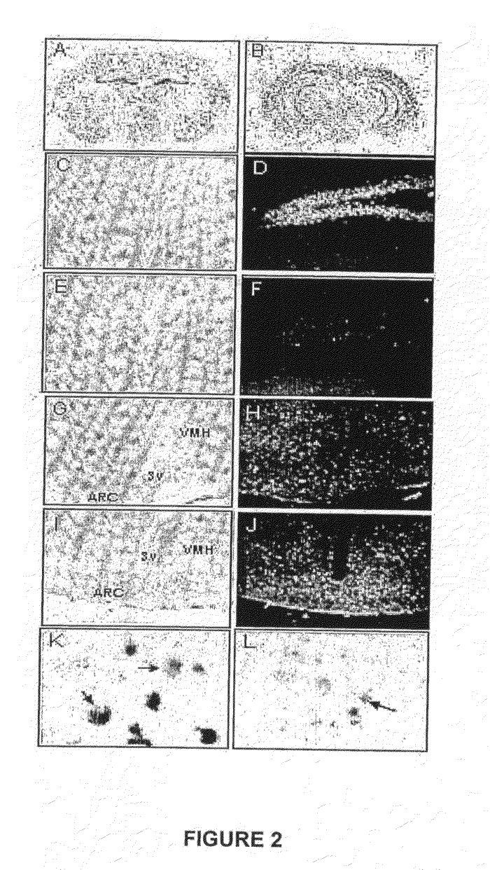

FIT mRNA was expressed throughout the CNS with highest levels seen in the hippocampus, amygdala, thalamic regions and hypothalamus including the arcuate nucleus (see FIG. 2). The arcuate nucleus is a hypothalamic region known to be the source and/or site of action of several neuropeptides that regulate energy balance. FIT mRNA was also observed to be co-localised with some POMC- as well as NPY-containing neurons in the arcuate nucleus of P. obesus. These data further support our assertion of a key role for FIT in the central regulation of energy balance.

### Example 14

### FIT Expression in Blood

FIT gene expression is greatest in the brain, with very small amounts found in most other tissues. The inventors attributed these small amounts to neural cells within the tissues. However slightly higher levels in the spleen, together with reports of melanocortin signalling in macrophages suggested that FIT may also be involved in the immune response and other aspects of macrophage function.

FIT gene expression was measured in human cord blood cells immunomagnetically sorted into T cells (CD3), B cells (CD19), NK cells (CD56) and monocytes/granulocytes (CD3/19/56 NEG). The monocyte/granulocyte sample contains approximately half monocytes and half granulocytes, and of the granulocytes approximately 75% are neutrophils and the remaining 25% are eosinophils, basophils etc. It is still not known exactly which cells within this sample express FIT, but because monocytes express both MC1R and MC3R, and given FITs previously implicated role in melanocortin signalling, it is likely that it is the monocyte population expressing FIT. Neutrophils have also been documented to express MC1R so FIT may also be expressed in these cells, and possibly the other granulocytes. Levels of FIT expression in T cells, B cells and NK cells was negligible.

TABLE 17
FIT gene expression in cord blood samples.

| Group | FIT gene expression (arbitrary units) |
|---|---|
| T cells | 0.3 ± 0.1 |
| B cells | 1.0 ± 0.8 |
| NK cells | 0.9 ± 0.7 |
| Monocytes and granulocytes | 10.4 ± 1.4^{a} |

(Mean ± SEM)
^{a}p < 0.024 compared to all other groups

α-MSH functions as a mediator of immunity and inflammation by downregulating the synthesis and release of proinflammatory cytokines such as IL-1, IL-6 and TNFα as well as the production of proinflammatory nitric oxide and neopterin by macrophages. In contrast, α-MSH upregulates the mRNA expression and release of the cytokine synthesis inhibitor IL-10 in monocytes. In addition to their pituitary and hypothalamic origin, POMC peptides have also been detected in several other organs and cells including epithelial cells, endothelial cells and immunocompetent cells. FIT gene expression is upregulated in a population of cells containing both monocytes and granulocytes and implicates FIT as possibly being involved in the regulation of melanocortin signalling in inflammation and immune responses. FIT is therefore also a novel target for therapeutics to enhance immune responses.

### BIBLIOGRAPHY

Altschul et al. (Nucl. Acids Res. 25: 3389, 1997.
Australian Institute of Health and Welfare (AIWH), Australia's Health, 2002, Canberra: AIWH.
Ausubel et al. “Current Protocols in Molecular Biology” John Wiley & Sons Inc, 1994-1998, Chapter 15.
Barnett et al., Diabetologia. 37: 671-676, 1994a.
Barnett et al., Diabete Nutr Metab 8: 42-47, 1995.
Barnett et al., Int. J Obesity 18: 789-794, 1994b.
Bertagna, Endocrinol Metab Clin North Am 23 (3): 467-485, 1994.
Bonner and Laskey, Eur. J. Biochem. 46: 83, 1974.
Bouchard, The genetics of Obesity, Boca Raton: CRC Press, 1994.
Branson et al., N Engl J Med 348 (12): 1096-1103, 2003.
Butler et al., Endocrinology 141 (9): 3518-3521, 2000.
Chen et al., Nat Genet 26 (1): 97-102, 2000.
Chevray and Nathans, Proc Natl Acad Sci USA 89 (13): 5789-5793, 1992.
Collier et al., Ann New York Acad Sci 827: 50-63, 1997a.
Collier et al., Exp Clin Endocrinol Diabetes 105: 36-37, 1997b.
Cummings and Schwartz, Nat Genet 26 (1): 8-9, 2000.
Dunstan et al., Diabetes Res. Clin. Pract. 57: 119-129, 2002.
Fan et al., Nature 385 (6612): 165-168, 1997.
Farooqi et al., J Clin Invest 106 (2): 271-279, 2000.
Farooqi et al., N Engl J Med 348 (12): 1085-1095, 2003.
Hinney et al., J Clin Endocrinol Metab 84 (4): 483-1486, 1999.
Hinney et al., J Clin Endocrinol Metab 88 (9): 4258-4267, 2003.
Ho and MacKenzie, J Biol Chem 274 (50): 35816-35822, 1999.
Huszar et al., Cell 88 (1): 131-141, 1997.
Jegou et al., J Neuroendocrinol 12 (6): 501-505, 2000.
Kask and Schioth, Brain Res 887 (2): 460-464, 2000.
Kopelman et al., Int. J Obesity 18: 188-191, 1994.
Kopelman, Nature 404: 635-643, 2000.
Krude and Gruters, Trends Endocrinol Metab 11 (1): 15-22, 2000.
Lubrano-Berthelier et al., Ann N Y Acad Sci 994: 49-57, 2003.
Marmur and Doty, J. Mol. Biol. 5: 109, 1962.
Marx, Science 686-689, 2002.
Mokdad et al., JAMA 282(16): 1519-1522, 1999.
Murphy et al., Neuropeptides 32 (6): 491-497, 1998.
Must et al., JAMA 282(16): 1523-1529, 1999.
National Health and Medical Research Council, Acting on Australia's weight: A strategy for the prevention of overweight and obesity. Canberra: National Health and Medical Research Council, 1996.
Nijenhuis et al., J Biol Chem 278 (25): 22939-22945, 2003.
Pritchard et al., J Endocrinol 172 (3): 411-421, 2002.
Ravussin, Metabolism 44(3): 12-14, 1995.
Risk Factor Prevalence Study Management Committee. Risk Factor Prevalence Study: Survey No. 3:1989. Canberra: National hearth Foundation of Australia and Australian Institute of Health, 1990
Vaisse et al., Nat Genet 20 (2): 113-114, 1998.
VanLeeuwen et al., J Biol Chem 278 (18): 15935-15940, 2003.
Vidal et al., Proc Natl Acad Sci USA 93 (19): 10321-10326, 1996.
Walder et al., Obesity Res 5: 193-200, 1997a.
Waters and Bennett, Risk Factors for Cardiovascular Disease: A Summary of Australian data. Canberra: Australian Institute of Health and Welfare, 1995.
World Trade Organisation. Obesity. Preventing and managing the global epidemic. Report of a WHO Consultation on Obesity. Geneva: World Health Organisation, 1998.
Yang et al., Mol Endocrinol 11 (3): 274-280, 1997.
Yaswen et al., Nat Med 5 (9): 1066-1070, 1999.
Yeo et al., Hum Mol Genet 12 (5): 561-574, 2003.
Yeo et al., Nat Genet 20 (2): 111-112, 1998.
Zimmet, Diabetes Care 15: 232-252, 1992.

1. An isolated compound which is a ligand of or otherwise interacts with a protein which comprises an amino acid sequence as set forth in SEQ ID NO: 2 or an amino acid sequence having at least about 40% similarity thereto.

2. The isolated compound of claim 1 wherein the protein is encoded by a nucleic acid molecule which comprises a nucleotide sequence as set forth in SEQ ID NO: 1 or a nucleotide sequence having at least about 40% identity thereto or a nucleotide sequence capable of hybridizing to SEQ ID NO: 1 or its complementary form under low stringency conditions.

3. The isolated compound of claim 2 wherein the compound is a ligand of FIT defined by amino acid sequence SEQ ID NO: 2.

4. The isolated compound of claim 2 wherein the compound is a ligand of FIT defined by an amino acid sequence encoded by FIT defined by nucleotide sequence SEQ ID NO: 1.

5. The isolated compound of claim 1 wherein the compound is a peptide, polypeptide or protein or a chemical analog, mimetic or homolog thereof.

6. The isolated compound of claim 1 wherein the compound is a chemical molecule.

7. The isolated compound of claim 1 wherein the compound is a soluble receptor for FIT.

8. The isolated compound of claim 1 wherein the compound is selected from the group consisting of endophilin 3, endophilin 1, β-arrestin 1, β-arrestin 2, the α-1 subunit of the AP2 complex, and the α-2 subunit of the AP2 complex, or a homolog, derivative or mimetic thereof.

9. An agonist or antagonist of the isolated compound of claim 1.

10. A method of identifying a ligand of the protein FIT or its derivatives, said method comprising introducing a first genetic construct in a yeast strain, said genetic construct comprising a nucleotide sequence encoding all or part of FIT fused to a nucleotide sequence encoding one of a DNA binding (DB) domain or an activation domain (AD) and introducing a second genetic construct into said yeast comprising a cDNA, said second genetic construct comprising elements of a cDNA library fused to a nucleotide sequence encoding the other of a DB domain or AD domain and selecting yeast cells which comprise both genetic constructs and in which a reporter gene has been subjected to two-hybrid dependent transcription.

11. The method of claim 10 wherein the yeast reporter gene is HI53.

12. A method for modulating expression of genetic material encoding a FIT ligand, selected from the group consisting of endophilin 3, endophilin 1, β-arrestin 1, β-arrestin 2, the α-1 subunit of the AP2 complex and the α-2 subunits of the AP2 complex, or a homologs or derivatives thereof in a mammal, said method comprising contacting the FIT ligand gene material with an effective amount of a modulator of the expression of the FIT ligand genetic material for a time and under conditions sufficient to up-regulate or down-regulate or otherwise modulate expression of the FIT ligand genetic material.

13. A method of modulating activity of FIT in a mammal, said method comprising administering to said mammal a modulating effective amount of a soluble FIT ligand or a derivative thereof or an antagonist or agonist of FIT-ligand interaction for a time and under conditions sufficient to increase or decrease FIT activity or levels.

14. A method of treating a mammal suffering from a condition characterized by one or more symptoms of an unhealthy state, including the presence or absence of a disorder associated with obesity, anorexia, weight maintenance, inflammation, diabetes, and/or metabolic energy levels comprising administering to said mammal an effective amount of an agent for a time and under conditions sufficient to modulate the activity of FIT or the interaction between FIT and a FIT ligand.

15. A composition comprising a FIT ligand or a soluble form of a FIT ligand selected from the group consisting of endophilin 3, endophilin 1, β-arrestin 1, β-arrestin 2, the α-1 subunit of the AP2 complex, and the α-2 subunits of the AP2 complex, or a modulator of a gene expression of a FIT ligand, or an antagonist or agonist of FIT-ligand interaction, and one or more pharmaceutically acceptable carriers and/or diluents.

16. A method of manufacture of a medicament comprising the isolated compound of claim 1 for the treatment of obesity, anorexia, weight maintenance, inflammation and/or metabolic energy levels.

17. The method of claim 16 wherein the protein is encoded by a nucleic acid molecule which comprises a nucleotide sequence as set forth in SEQ ID NO: 1 or a nucleotide sequence having at least about 40% identity thereto or a nucleotide sequence capable of hybridizing to SEQ ID NO: 1 or its complementary form under low stringency conditions.

18. The method of claim 17 wherein the compound is a ligand of FIT defined by amino acid SEQ ID NO: 2.

19. The method of claim 17 wherein the compound is a ligand of FIT defined by an amino acid sequence encoded by FIT defined by nucleotide sequence SEQ ID NO: 1.

20. The method of claim 17 wherein the isolated compound of claim 1 is a peptide, polypeptide or protein or a chemical analog, mimetic or homolog thereof.

21. The method of claim 17 wherein the isolated compound of claim 1 is a chemical molecule.

22. The method of claim 17 wherein the isolated compound of claim 1 is a soluble receptor for FIT.

23. The method of claim 17 wherein the isolated compound of claim 1 is selected from the group consisting of endophilin 3, endophilin 1, β-arrestin 1, β-arrestin 2, the α-1 subunit of the AP2 complex and the α-2 subunit of the AP2 complex, or a homolog, derivative or mimetic thereof.


SEQUENCE LISTING (67 sequences)

SEQ ID NO: 1 - DNA, 6317 bp, Psammomys obesus
cagactccttggaaattaaggaatgcaattctgccaccatgatggaaggactgaaaaaacgtacaaggaaggcctttggaatacggaagaaagaaaaagacactgactctacaggctcaccagatcgagatggaatgcagcccagcccacacgagctcccctaccatagcaaagcagagtgtgcccgagaaggagggaacaaagcttcgaagaaaagcaatggggcaccaaatggattttatgcggaaattgattgggaaagatataactcacctgagctggatgaagaaggttacagcatcagacctgaggaaccaggctctaccaaaggaaagcacttttattcttcaagtgaatccgaagaggaggaagaatcgcacaagaagttcaatatcaagattaaacccttgcagtccaaggacatccttaagaatgctgcaacagtagacgagctgaaggcttccataggcaacattgcactttccccttcgcctgtgaggaaaagtccgaggcgcagcccgggtgcaattaaaaggaacttatccagtgaagaagtcgcaagacccaggcgttccaccccaactccagaacttacaagcaagaagcctctggacgacactctggcccttgctcccctctttggcccaccgttagaatctgcttttgatggacacaagacggaagttcttttagatcagcctgagatatggggttcaggccaaccagttaacccaagcatggagtcaccaaagctagcaagaccttttcccactggaacccctccacctctgcctccaaaaactgtaccagccaccccgcctcggacaggctcccccttaacagtggcgacaggaaatgaccaggcagccacagaggccaaaattgagaaactaccatccatcagtgacctggacagcatttttggccccgtgttgtcccccaagtctgttgctgttaatactgaggagacgtgggtccatttctctgatgcatccccggaacatgttactccagagttgactccaagggaaaaggtggtgaccccaccagctgcatcagacatcccagctgactccccaactccaggcccgcctggccccccaggctcggcaggtcccccagggcctcctggtcctcgcaatgtaccatctccgctcaatttagaagaagtccagaagaaagtcgctgagcagaccttcattaaagatgattacttagaaacactctcatctcctaaagagtgtgggttgggacagagagcaactccacctcccccaccaccacccacctacaggactgtggtttcgtcccccggacctggctcgggcagtggtacggggaccgccagtggtgcatcgtcccctgctcggccagccacccccttagttccttgcagctgctccactccgcctccacctcctccccggcctccatcccggccaaagctacctccaggaaagcctggagttggagacgtgtccagaccttttagcccacccatacactcctccagccctcctccaatagcacccttagcccgggctgaaagcacttcttcaatatcatcaaccaattccctgagcgcagccaccactcccacagttgagaatgaacagscttccctcgtttggtttgacagaggaaagttttatttgacttttgaaggttcttccaggggacccagtcctctaactatgggggcccaggacaccctcccggttgcagcagcattcacagaaactgtcaatgcctacttcaaaggagcagatccaagcaaatgcattgttaagatcacgggagaaatggtgttgtcctttcctgctggcatcaccagacactttgccaacaacccatccccagctgctctgacttttcgagtgataaattccagcaggttagagcacgtcctgccgaacccccagctcctctgctgcgataacacacaaaatgatgccaataccaaggaattctgggtaaacatgccaaatttgatgacccacctgaagaaggtctctgaacaaaaaccccaggctacatattacaatgtggacatgctcaagtatcaggtgtcagcccagggcattcagtccacacctctgaacttggcggtgaactggcgctgtgagccttccagcactgacctgcgcatagattataagtacaacacggatgccatgtccaccgcagtggcccttaacaacgtgcagttcctggtccccattgatggaggagtgaccaagctccaggctgtccttcctccagcagtctggaatgctgaacaacaaagaatattatggaagattcctgatatctcccagaagtcagaaaatggaggcgtaggttctttactggcaagatttcaattagccgaaggcccaagcaaaccttccccactggtcgtgcagttcacgagtgaagggagcactctgtctggctgcgacattgagcttgtcggagcagggtacgggttttcactcatcaagaagaggtttgctgcaggaaaatacttggccgataactaataaaatgtcatgcaaggattttgaagatccatgtcctggagaactgttgtctgagagacatattttaatctggtttgaggaaaacaaaccaaccgatgtctgtacgtgggctctgtcagctggaaggtcccggctttcagccgtgatttcccacacccagtacaaggaggatcagttctacagtacttacttctaggtgtactattgttaatggttttaaaatgtaattattgtatttgtaaactgtaccttcattccagtaaggcagttagacacctgagttttagcttttttttccattcctgaaacggatgtaatttaaactgcggtatgtaaatttaatagtagtactgtcgaatggcacaatgcttacagagatacagtgcattttgtcaatatataaaatttaaatataatgttgatagttaccataaagggggtgccacacatcaagaaccttaaatggaaccagaaacaagcaagcaaacaaacaaacaaacaaacaaaaccttacttttcttcactccttattacattttcctctagagctaaagaaacttctagcttcggtttagtgggttaaattcagaaactatttcagaaaaaaaaaaaaattctgaagttacagcatattcaaagagaagcattaattaccacttttttaaaagcttttttttcaaaccgcaaatttcataaaaatgcaaactgtgtaaacagggcctcttatttttataacttgtgtaaaaagggaaaatcaattcatatttaaagtttaagtagtattaaattatatccaagagtgaagaggatgttgaaatcttacctgaccccatgccccttctttgcagtttagcaaatgttgagattgctaaatcatcagattaaagccaacttgatttttaaagtttcaagactttctgaagctgaactggttaaaacttttgcacaattgcttggaacggagggggaggggcctctctggtccagcacaggtaccttgtttcttccctactcacaagaatcaaaacaatgaaagtcaagaaccacagaggggggaaattagttccctgttcagtccaaaaggagaactttaaacttatcatttacgtctttggggaaggaagaaataagctttataagtgaaatcctattcaccttgttgtcctatgaatgttttcggggtgactttaagattcattgtatacatgtgcgagtctctgctattcttggggagttgaaagcagagccaggccagtggccttgaagttcagtaaatgccacagttctggggcaaaggtaggcatgagggttctgcccctcagcacaggaatcagagcagtgtcttgtaaggtctaaagattaagtcttccagtaagccacaagttattttgtaacagagttggggagttttggcactcgctgctgactttcattttgtatccactcaaatggagtcttcaactcttttcaactttagaatcaaattaatttttttttttttttttttttttttacacaaggtttactctgtgtaactgtcctggatgttctggaactctttttgtagaccaggctggcctcgaactcagagagatccacctgcctgtgctccccaagtgctgggattaaaggcgtgtgccaccatgcctggcttagattaaattttttaagtcttacttcaccagtgagattgtgattggcagttgtttcgagagagctttgtagcttaatctatgttctcttcaatcaatgcttgctaccaaaagaatgtccaaaatgatctatttttcctgggaacaattcatctatttaaataggctcttgcctagttccccaaagcagcctgtctttgaaggtttttttgaacaaaataattttttcacaaaaagtttggttttgaaatcaaaatagagaaataaaatgtaaattttaaatctaatggaacatgaggaaatgaaaaaacttaagccaatggagagtaaaagcagaaaaaaatgaaacttacctagaatgtgattatattatgtttttaagtagtcaattcatggaaaaatattgaatattaacacaaagcatattaaaaatatgtaaatattactgtttctcatgtctttctctttatatcttattttatatagttttagaatgaattggtcattaaatacagtgtttctttccaaagaataattttgttgatattgtaaaaatgtaattaaagatagagacttgaatagtctctaacattatccaaatgtttctaggaaccaaattcaaagctgtgaagaaagcttgcaatccctgaattggcttttgtgaaatggaatgacggtgggtaatctcaaaattcagacttgaatagtcagagctgaagtggggaatgggtggttccttctggttcagaaaataggtcaaataacagcatttgctcgcatcagggatggagatgttggtgatgtttggttttactctcgcaggctttcgtctcctgttgaaggtgtatctgtagcccagtgggataagagttcatgttctgagatgtggtcctagacaaggcaggcaaggtttcagtcatcaatacctatcaggtcaggttcccttttgtctatacaaaatgggttagctcatagccagatggtttgcaggacagtgagctaaattaggacaagattctggttagccaaagagctgtttcctaagcactctgatttttttttaaagctgatagaaagtgtaaatgttctattttgacgacatggaaagtatgttttcctcttcaaataaatcccttatttttatgaaattttcaaaaataaattcttgtttaaaatagtctgaatgttatcatagttggaacttggcaattactaatttgaaattctatgagatgtatctccagctaaaatggcaattccctgtatgctatctggggctcagtttacctctaaggaagactgtcagagtgcaaatggttttgagtgacgggaaagtcaaagggcaaatgtttgtgcttttttctttttctgtcttatatacttcttcttggtctcagaatgcaaagtatcagagccatagttacacacatttccacttttaacgcttcttttgaaggaagcagatccacttttgccccgccactcatgcctgctgtgcagactcagacgagtccctgccctcttcacgcctttggggtgagaggggagccatatgtaagtagttttcaagcttttcttaatgggacttttctttttctaataaaatcatgcctggaatcctgtaaagattgttgcctggctgtgaaggggcttctccagatcctgaaatatagcatcacaatacgtaaatgactcccgatggatctcccagctctgaagacttgctcttctacttcacatgtgtagccacgacgatcagctggcacacagtacaattagctgtgtagtgagtgctccccagctatcagtcatgaaacatatcactttgctcaacctgtttttaaaaaagctccaaaatggtaaaaatgcttttcagtctttgttttcccaataatggtattgaggcctaagctgattaacttcccccaaagtggtaccacagctggtaacgaccccaatgatcctgaaaaaaatggaatgagtaccttgctgtttcrtttagttyattttgggaaaataatccatttgaatgtcaagataaaaaggcaccaggaaaagtcctcattggaaggattaaagatgagcctggtaagatgttaagatgtaagatgttaagatgtgttactgtaaaaaaaaaaagctt

SEQ ID NO: 2 - protein, 827 aa, Psammomys obesus
MMEGLKKRTRKAFGIRKKEKDTDSTGSPDRDGMQPSPHELPYHSKAECAREGGNKASKKSNGAPNGFYAEIDWERYNSPELDEEGYSIRPEEPGSTKGKHFYSSSESEEEEESHKKFNIKIKPLQSKDILKNAATVDELKASIGNIALSPSPVRKSPRRSPGAIKRNLSSEEVARPRRSTPTPELTSKKPLDDTLALAPLFGPPLESAFDGHKTEVLLDQPEIWGSGQPVNPSMESPKLARPFPTGTPPPLPPKTVPATPPRTGSPLTVATGNDQAATEAKIEKLPSISDLDSIFGPVLSPKSVAVNTEETWVHFSDASPEHVTPELTPREKVVTPPAASDIPADSPTPGPPGPPGSAGPPGPPGPRNVPSPLNLEEVQKKVAEQTFIKDDYLETLSSPKECGLGQRATPPPPPPPTYRTVVSSPGPGSGSGTGTASGASSPARPATPLVPCSCSTPPPPPPRPPSRPKLPPGKPGVGDVSRPFSPPIHSSSPPPIAPLARAESTSSISSTNSLSAATTPTVENEQASLVWFDRGKFYLTFEGSSRGPSPLTMGAQDTLPVAAAFTETVNAYFKGADPSKCIVKITGEMVLSFPAGITRHFANNPSPAALTFRVINSSRLEHVLPNPQLLCCDNTQNDANTKEFWVNMPNLMTHLKKVSEQKPQATYYNVDMLKYQVSAQGIQSTPLNLAVNWRCEPSSTDLRIDYKYNTDAMSTAVALNNVQFLVPIDGGVTKLQAVLPPAVWNAEQQRILWKIPDISQKSENGGVGSLLARFQLAEGPSKPSPLVVQFTSEGSTLSGCDIELVGAGYGFSLIKKRFAAGKYLADN

SEQ ID NO: 3 - protein, 499 aa, Human
MMEGLKKRTRKAFGIRKKEKDTDSTGSPDRDGMQPSPHELPYHSKAECAREGGKKASKKSNGAPNGFYAEIDWERYNSPELDEEGYSIRPEEPGSTKGKHFYSSSESEEEEESHKKFNIKIKPLQSKDILKNAATVDELKASIGNIALSPSPVRKSPRRSPGAIKRNLSSEEVARPRRSTPTPELTSKKPLDDTLALAPLFGPPLESAFDGHKTEVLLDQPEIWGSGQPVNPSMESPKLARPFPTGTPPPLPPKTVPATPPRTGSPLTVATGNDQAATEAKIEKPPSISDLDSIFGPVLSPKSVAVNTEETWVHFSDASPEHVTPELTPREKVVTPPAASDIPADSPTPGPPGPPGSAGPPGPPGPRNVPSPLNLEEVQKKVAEQTFIKDDYLETLSSPKECGLGQRETPPPPPPPTYRTVVSSPGPGSGSGTGTASGASSPARPATPLVPCSCSTPPPPPPRPPSRPKLPPGKPGVGDVSRPFSPPIHSSSPPPIAPL

SEQ ID NO: 4 - protein, 259 aa, Human
RPFPTGTPPPLPPKTVPATPPRTGSPLTVATGNDQAATEAKIEKPPSISDLDSIFGPVLSPKSVAVNTEETWVHFSDASPEHVTPELTPREKVVTPPAASDIPADSPTPGPPGPPGSAGPPGPPGPRNVPSPLNLEEVQKKVAEQTFIKDDYLETLSSPKECGLGQRATPPPPPPPTYRTVVSSPGPGSGSGTGTASGASSPARPATPLVPCSCSTPPPPPPRPPSRPKLPPGKPGVGDVSRPFSPPIHSSSPPPIAPL

SEQ ID NO: 5 - DNA, 21 bp, Artificial Sequence
  SNP ID 1373910 Forward primer
gttgttctatcactggcagtc

SEQ ID NO: 6 - DNA, 21 bp, Artificial Sequence
  SNP ID 1373910 Reverse primer
gagcattgcaaagaggatggg

SEQ ID NO: 7 - DNA, 37 bp, Artificial Sequence
  SNP ID 1373910 SNP sequence
caaagaggatgggagggtttntattaggtcctgtgtg

SEQ ID NO: 8 - DNA, 21 bp, Artificial Sequence
  SNP ID 1445579 Forward primer
gcaagcaaactgcagcatttc

SEQ ID NO: 9 - DNA, 21 bp, Artificial Sequence
  SNP ID 1445579 Reverse primer
gatgtgcagccagtgtatgtg

SEQ ID NO: 10 - DNA, 41 bp, Artificial Sequence
  SNP ID 1445579 SNP sequence
ttctagaacctggttgccaangttttgcaagcagaaatgct

SEQ ID NO: 11 - DNA, 21 bp, Artificial Sequence
  SNP 1900105 Forward primer
gttcttccctctgggtctatc

SEQ ID NO: 12 - DNA, 21 bp, Artificial Sequence
  SNP 1900105 Reverse primer
gaataaggaaaggcctccagc

SEQ ID NO: 13 - DNA, 42 bp, Artificial Sequence
  SNP 1900105 SNP sequence
tgggtctatctcctgctctgtgnctttacctctggtcacagg

SEQ ID NO: 14 - DNA, 21 bp, Artificial Sequence
  SNP 2146904 Forward primer
gaactgtcatgcaacctgctg

SEQ ID NO: 15 - DNA, 21 bp, Artificial Sequence
  SNP 2146904 Reverse primer
gctcagatgcaccctgtatat

SEQ ID NO: 16 - DNA, 38 bp, Artificial Sequence
  SNP 2146904 SNP sequence
tgtatatttactgttcatcntggaactcgtgccactga

SEQ ID NO: 17 - DNA, 21 bp, Artificial Sequence
  SNP 4143026 Forward primer
gcaaattagcctgccagagag

SEQ ID NO: 18 - DNA, 21 bp, Artificial Sequence
  SNP 4143026 Reverse primer
gtgaagtgaggacaggaaagg

SEQ ID NO: 19 - DNA, 41 bp, Artificial Sequence
  SNP 4143026 SNP sequence
gcaaaattcatgaatttgcngctgcttggtaacaccacccc

SEQ ID NO: 20 - DNA, 21 bp, Artificial Sequence
  SNP 604737 Forward primer
ggtgccattcaacaatactac

SEQ ID NO: 21 - DNA, 21 bp, Artificial Sequence
  SNP 604737 Reverse primer
gggcagttagacacttgagtt

SEQ ID NO: 22 - DNA, 43 bp, Artificial Sequence
  SNP 604737 SNP sequence
ttttaccattcctgaaatggatntaatttaaactgtggtatgt

SEQ ID NO: 23 - DNA, 21 bp, Artificial Sequence
  SNP 485521 Forward primer
ggtaaaaagggaaagcaattc

SEQ ID NO: 24 - DNA, 21 bp, Artificial Sequence
  SNP 485521 Reverse primer
ggagaggggcaagtagttaag

SEQ ID NO: 25 - DNA, 41 bp, Artificial Sequence
  SNP 485521 SNP sequence
tcaagagtaaagaagatgntgaagtcttaactacttgcccc

SEQ ID NO: 26 - DNA, 21 bp, Artificial Sequence
  SNP 1373909 Forward primer
gctcccatcctctttgcaatg

SEQ ID NO: 27 - DNA, 21 bp, Artificial Sequence
  SNP 1373909 Reverse primer
gttctgcttagaaggcttggg

SEQ ID NO: 28 - DNA, 41 bp, Artificial Sequence
  SNP 1373909 SNP sequence
gtgttcatggagataacagcnaatggtcttccaggaattta

SEQ ID NO: 29 - DNA, 21 bp, Artificial Sequence
  SNP 4655650 Forward primer
gtgcaggcgttttcagttttg

SEQ ID NO: 30 - DNA, 21 bp, Artificial Sequence
  SNP 4655650 Reverse primer
gcagacattaaccccatgaac

SEQ ID NO: 31 - DNA, 42 bp, Artificial Sequence
  SNP 4655650 SNP sequence
cgttttcagttttgaagcatattnataggaggctttaaatca

SEQ ID NO: 32 - DNA, 21 bp, Artificial Sequence
  SNP 657808 Forward primer
gtaaaactctccttctggatc

SEQ ID NO: 33 - DNA, 21 bp, Artificial Sequence
  SNP 657808 Reverse primer
gacccacaggaatcaaaacgc

SEQ ID NO: 34 - DNA, 40 bp, Artificial Sequence
  SNP 657808 SNP sequence
aattttagggaaaaaaaagtccnctgtttagatccagaag

SEQ ID NO: 35 - DNA, 21 bp, Artificial Sequence
  SNP 1373911 Forward Primer
gccccatttcatttggccaac

SEQ ID NO: 36 - DNA, 21 bp, Artificial Sequence
  SNP 1373911 Reverse primer
gtttggggatgcatctacaag

SEQ ID NO: 37 - DNA, 41 bp, Artificial Sequence
  SNP 1373911 SNP sequence
tcttaaatttacttrgccttangtttagatccaacttggat

SEQ ID NO: 38 - DNA, 21 bp, Artificial Sequence
  SNP 2146905 Foward primer
gtctcacatgcagccacaaag

SEQ ID NO: 39 - DNA, 21 bp, Artificial Sequence
  SNP 2146905 Reverse primer
gtgctccccagaaaattggtc

SEQ ID NO: 40 - DNA, 40 bp, Artificial Sequence
  SNP 2146905 SNP sequence
taattcattcatttgagagacnctaaaggaaggaaaattg

SEQ ID NO: 41 - DNA, 22 bp, Artificial Sequence
  SNP 4655643 Forward primer
ggggtgggtagttttaaatgtc

SEQ ID NO: 42 - DNA, 21 bp, Artificial Sequence
  SNP 4655643 Reverse primer
gactattttccgttactctcc

SEQ ID NO: 43 - DNA, 40 bp, Artificial Sequence
  SNP 4655643 SNP sequence
tacrttttcctataaactcntcatgtggagagtaacggaa

SEQ ID NO: 44 - DNA, 21 bp, Artificial Sequence
  SNP 1338200 Forward primer
gatgaactgcagaggcagtac

SEQ ID NO: 45 - DNA, 21 bp, Artificial Sequence
  SNP 1338200 Reverse primer
gttttccaaatgaaaatacag

SEQ ID NO: 46 - DNA, 39 bp, Artificial Sequence
  SNP 1338200 SNP sequence
tgaaaatacagagcgaganagctttttttaaaaaaaata

SEQ ID NO: 47 - DNA, 21 bp, Artificial Sequence
  SNP 502690 Forward primer
gccccaagaacctcaggaaat

SEQ ID NO: 48 - DNA, 21 bp, Artificial Sequence
  SNP 502690 Reverse primer
gtacttttcagagcaaagcac

SEQ ID NO: 49 - DNA, 42 bp, Artificial Sequence
  SNP 502690 SNP sequence
tttaaataataaaaatgatgtntatatgtgtgctttgctctg

SEQ ID NO: 50 - DNA, 21 bp, Artificial Sequence
  SNP 3078564 Forward primer
ggattcagtgtattgacatgg

SEQ ID NO: 51 - DNA, 21 bp, Artificial Sequence
  SNP 3078564 Reverse primer
gtgacaacaccatttctccgg

SEQ ID NO: 52 - DNA, 40 bp, Artificial Sequence
  SNP 3078564 SNP sequence
gtattgacatggattttctctcntttcctctctgtgtttt

SEQ ID NO: 53 - DNA, 21 bp, Artificial Sequence
  SNP 1325267 Forward primer
gtgctgaatgacagtttgccc

SEQ ID NO: 54 - DNA, 21 bp, Artificial Sequence
  SNP 1325267 Reverse primer
gatggagcagaagtcttcctg

SEQ ID NO: 55 - DNA, 39 bp, Artificial Sequence
  SNP 1325267 SNP sequence
gtgcagttaaaatatgctgangcccctgcatggccagga

SEQ ID NO: 56 - DNA, 21 bp, Artificial Sequence
  SNP 1856319 Forward primer
gccaacttccttttgtagagc

SEQ ID NO: 57 - DNA, 21 bp, Artificial Sequence
  SNP 1856319 Reverse primer
gttagatgtggaaaacttgcc

SEQ ID NO: 58 - DNA, 39 bp, Artificial Sequence
  SNP 1856319 SNP sequence
aatcaaggggaaagaaaaanttgaattgctctacaaaag

SEQ ID NO: 59 - DNA, 21 bp, Artificial Sequence
  SNP 1325266 Forward primer
ggggtgttttgtgtctggatg

SEQ ID NO: 60 - DNA, 21 bp, Artificial Sequence
  SNP 1325266 Reverse primer
gcagggaagatgtcacatatc

SEQ ID NO: 61 - DNA, 39 bp, Artificial Sequence
  SNP 1325266 SNP sequence
ggatgcctaaggtgattccangggaggggatggaagata

SEQ ID NO: 62 - DNA, 41 bp, Artificial Sequence
  SNP 3078564 SNP sequence
gtattgacatggattttctctccntttcctctctgtgtttt

SEQ ID NO: 63 - DNA, 35 bp, Artificial Sequence
  FIT-NP oligonucleotide
gtacagtcgactatgatggaaggactgaaaaaacg

SEQ ID NO: 64 - DNA, 28 bp, Artificial Sequence
  FIT-PR oligonucleotide
gtacagtcgaccagaccttttcccactg

SEQ ID NO: 65 - DNA, 26 bp, Artificial Sequence
  Antisense oligonucleotide
atagcggccgcggctaagggtgctat

SEQ ID NO: 66 - DNA, 21 bp, Artificial Sequence
  oligonucleotide primer
tgaaggcttccataggcaaca

SEQ ID NO: 67 - DNA, 18 bp, Artificial Sequence
  oligonucleotide primer
tggaacgcctgggtcttg